SEQUENCE LISTING (61 sequences)

SEQ ID NO: 1 - DNA, 21 bp, Artificial Sequence
  RHO17-SgRNA1
cgtaccacacccatcgcattg

SEQ ID NO: 2 - DNA, 21 bp, Artificial Sequence
  RHO17-SgRNA2
tgcgtaccacacccatcgcat

SEQ ID NO: 3 - DNA, 21 bp, Artificial Sequence
  RHO17-SgRNA3
ctgcgtaccacacccatcgca

SEQ ID NO: 4 - DNA, 21 bp, Artificial Sequence
  RHO135-SgRNA1
caccacgtaccactcgatggc

SEQ ID NO: 5 - DNA, 21 bp, Artificial Sequence
  RHO135-SgRNA2
cactcgatggccaggaccacc

SEQ ID NO: 6 - DNA, 60 bp, Artificial Sequence
  RHO17 SgRNA1-T7 forward primer
taatacgactcactatagcgtaccacacccatcgcattggttttagtactctggaaacag

SEQ ID NO: 7 - DNA, 60 bp, Artificial Sequence
  RHO17 SgRNA2-T7 forward primer
taatacgactcactatagtgcgtaccacacccatcgcatgttttagtactctggaaacag

SEQ ID NO: 8 - DNA, 60 bp, Artificial Sequence
  RHO17 SgRNA3-T7 forward primer
taatacgactcactatagctgcgtaccacacccatcgcagttttagtactctggaaacag

SEQ ID NO: 9 - DNA, 60 bp, Artificial Sequence
  RHO135 SgRNA1-T7 forward primer
taatacgactcactatagcaccacgtaccactcgatggcgttttagtactctggaaacag

SEQ ID NO: 10 - DNA, 60 bp, Artificial Sequence
  RHO135 SgRNA2-T7 forward primer
taatacgactcactatagcactcgatggccaggaccaccgttttagtactctggaaacag

SEQ ID NO: 11 - DNA, 22 bp, Artificial Sequence
  Reverse primer
atctcgccaacaagttgacgag

SEQ ID NO: 12 - DNA, 23 bp, Artificial Sequence
  RHO17-forward primer 1
ttaggagggggaggtcactttat

SEQ ID NO: 13 - DNA, 22 bp, Artificial Sequence
  RHO17-reverse primer 1
aaatccacttcccaccctgagc

SEQ ID NO: 14 - DNA, 21 bp, Artificial Sequence
  RHO135-forward primer 1
gcccagagcgctaagcaaata

SEQ ID NO: 15 - DNA, 20 bp, Artificial Sequence
  RHO135-reverse primer 1
atcgcctagaggctgagtgg

SEQ ID NO: 16 - DNA, 30 bp, Artificial Sequence
  RHO17-mKate-mut-sgRNA1 forward primer
ctagcgtaccacacccatcgcattggagaa

SEQ ID NO: 17 - DNA, 30 bp, Artificial Sequence
  RHO17-mKate-mut-sgRNA1 reverse primer
ggccttctccaatgcgatgggtgtggtacg

SEQ ID NO: 18 - DNA, 30 bp, Artificial Sequence
  RHO17-mKate-wt-sgRNA1 forward primer
ctagcgtaccacacccgtcgcattggagaa

SEQ ID NO: 19 - DNA, 30 bp, Artificial Sequence
  RHO17-mKate-wt-sgRNA1 reverse primer
ggccttctccaatgcgacgggtgtggtacg

SEQ ID NO: 20 - DNA, 30 bp, Artificial Sequence
  RHO17-mKate-mut-sgRNA2 forward primer
ctagtgcgtaccacacccatcgcattggag

SEQ ID NO: 21 - DNA, 30 bp, Artificial Sequence
  RHO17-mKate-mut-sgRNA2 reverse primer
ggccctccaatgcgatgggtgtggtacgca

SEQ ID NO: 22 - DNA, 30 bp, Artificial Sequence
  RHO17-mKate-wt-sgRNA2 forward primer
ctagtgcgtaccacacccgtcgcattggag

SEQ ID NO: 23 - DNA, 30 bp, Artificial Sequence
  RHO17-mKate-wt-sgRNA2 reverse primer
ggccctccaatgcgacgggtgtggtacgca

SEQ ID NO: 24 - DNA, 30 bp, Artificial Sequence
  RHO17-mKate-mut-sgRNA3 forward primer
ctagctgcgtaccacacccatcgcattgga

SEQ ID NO: 25 - DNA, 30 bp, Artificial Sequence
  RHO17-mKate-mut-sgRNA3 reverse primer
ggcctccaatgcgatgggtgtggtacgcag

SEQ ID NO: 26 - DNA, 30 bp, Artificial Sequence
  RHO17-mKate-wt-sgRNA3 forward primer
ctagctgcgtaccacacccgtcgcattgga

SEQ ID NO: 27 - DNA, 30 bp, Artificial Sequence
  RHO17-mKate-wt-sgRNA3 reverse primer
ggcctccaatgcgacgggtgtggtacgcag

SEQ ID NO: 28 - DNA, 30 bp, Artificial Sequence
  RHO135-mKate-mut-sgRNA1 forward primer
ctagcaccacgtaccactcgatggccagga

SEQ ID NO: 29 - DNA, 30 bp, Artificial Sequence
  RHO135-mKate-mut-sgRNA1 reverse primer
ggcctcctggccatcgagtggtacgtggtg

SEQ ID NO: 30 - DNA, 30 bp, Artificial Sequence
  RHO135-mKate-wt-sgRNA1 forward primer
ctagcaccacgtaccgctcgatggccagga

SEQ ID NO: 31 - DNA, 30 bp, Artificial Sequence
  RHO135-mKate-wt-sgRNA1 reverse primer
ggcctcctggccatcgagcggtacgtggtg

SEQ ID NO: 32 - DNA, 30 bp, Artificial Sequence
  RHO135-mKate-mut-sgRNA2 forward primer
ctagcactcgatggccaggaccaccaagga

SEQ ID NO: 33 - DNA, 30 bp, Artificial Sequence
  RHO135-mKate-mut-sgRNA2 reverse primer
ggcctccttggtggtcctggccatcgagtg

SEQ ID NO: 34 - DNA, 30 bp, Artificial Sequence
  RHO135-mKate-wt-sgRNA2 forward primer
ctagcgctcgatggccaggaccaccaagga

SEQ ID NO: 35 - DNA, 30 bp, Artificial Sequence
  RHO135-mKate-wt-sgRNA2 reverse primer
ggcctccttggtggtcctggccatcgagcg

SEQ ID NO: 36 - DNA, 21 bp, Artificial Sequence
  TS-SP001
ctgataggcagcctgcacctg

SEQ ID NO: 37 - DNA, 317 bp, Artificial Sequence
  Vector sequence I
ctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatgtaatctag

SEQ ID NO: 38 - DNA, 605 bp, Artificial Sequence
  Vector sequence II
ccgctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgacccgggtacccagctttcttgtacaaagtggtacgcgtgaattcactcctcaggtgcaggctgcctatcag

SEQ ID NO: 39 - DNA, 5 bp, Artificial Sequence
  PAM1
gagaa

SEQ ID NO: 40 - DNA, 5 bp, Artificial Sequence
  PAM2
tggag

SEQ ID NO: 41 - DNA, 5 bp, Artificial Sequence
  PAM3
ttgga

SEQ ID NO: 42 - DNA, 5 bp, Artificial Sequence
  PAM4
cagga

SEQ ID NO: 43 - DNA, 5 bp, Artificial Sequence
  PAM5
aagga

SEQ ID NO: 44 - RNA, 21 nt, Artificial Sequence
  RHO17-SgRNA1
cguaccacacccaucgcauug

SEQ ID NO: 45 - RNA, 21 nt, Artificial Sequence
  RHO17-SgRNA2
ugcguaccacacccaucgcau

SEQ ID NO: 46 - RNA, 21 nt, Artificial Sequence
  RHO17-SgRNA3
cugcguaccacacccaucgca

SEQ ID NO: 47 - RNA, 21 nt, Artificial Sequence
  RHO135-SgRNA1
caccacguaccacucgauggc

SEQ ID NO: 48 - RNA, 21 nt, Artificial Sequence
  RHO135-SgRNA2
cacucgauggccaggaccacc

SEQ ID NO: 49 - DNA, 22 bp, Artificial Sequence
  RHO17-forward primer2
ttatgaacacccccaatctccc

SEQ ID NO: 50 - DNA, 22 bp, Artificial Sequence
  RHO17-reverse primer2
gagggctttggataacattgac

SEQ ID NO: 51 - DNA, 21 bp, Artificial Sequence
  RHO17-forward primer3
ctagtgtcaccttggcccctc

SEQ ID NO: 52 - DNA, 21 bp, Artificial Sequence
  RHO17-reverse primer3
tgctgcaaacatggcccgaga

SEQ ID NO: 53 - DNA, 20 bp, Artificial Sequence
  RHO135-forward primer2
tggctcctaggagaggcccc

SEQ ID NO: 54 - DNA, 20 bp, Artificial Sequence
  RHO135-reverse primer2
ctgtttcttcttctgcccta

SEQ ID NO: 55 - DNA, 25 bp, Artificial Sequence
  WT-F
ggcagcagtgggattagcgttagta

SEQ ID NO: 56 - DNA, 25 bp, Artificial Sequence
  WT-R
tgtgtagagggtggtggtgaatcct

SEQ ID NO: 57 - DNA, 25 bp, Artificial Sequence
  Mut-R
acgatcagcagaaacatgtaggcgg

SEQ ID NO: 58 - DNA, 20 bp, Artificial Sequence
  RHO17-forward primer4
gagtgtggggactggatgac

SEQ ID NO: 59 - DNA, 20 bp, Artificial Sequence
  RHO17-reverse primer4
gggactctcccagacccctc

SEQ ID NO: 60 - DNA, 22 bp, Artificial Sequence
  RHO135-forward primer3
tgtccgggttatttcatttccc

SEQ ID NO: 61 - DNA, 20 bp, Artificial Sequence
  RHO135-reverse primer3
gagatgggaccagcccttgt

(19) United States
(12) Patent Application Publication
(10) Pub. No.: US 2023/0149439 A1
(43) Pub. Date: May 18, 2023

(54) RHO-ADRP GENE EDITING-BASED METHODS AND COMPOSITIONS

(71) Applicant: CHIGENOVO CO., LTD., Changping District, Beijing (CN)

(72) Inventors: Liping Yang, MI (US); Xiaozhen LIU, Västra Frölunda, CA (US); Jing Qiao, Beijing (CN); Fan Zhang, Beijing (CN); Tianfu Zhang, Beijing (CN); Saichao He, Beijing (CN); Luying Zeng, Beijing (CN); Hongjie Pei, Beijing (CN)

(21) Appl. No.: 17/996,786

(22) PCT Filed: Jul. 30, 2020
(86) PCT No.: PCT/CN2020/105881

§ 371 Date: Oct. 20, 2022

(30) Foreign Application Priority Data
Apr. 21, 2020 (CN) 202010318429.0

(51) Int. Cl. A61K 31/7105; A61P 27/02; A61K 38/46

(52) U.S. Cl. CPC A61K 31/7105; A61P 27/02; A61K 38/465

(57) ABSTRACT

The subject application relates to a method for treating retinitis pigmentosa, comprising the step of: enabling a subject in need thereof to have a functional RHO gene, wherein the functional RHO gene does not comprise a mutation site selected from the group consisting of c.C50T and c.C403T. The subject application also relates to a method for editing the RHO gene, and a composition for treating retinitis pigmentosa in a subject.

### TECHNICAL FIELD

The present application relates to the field of biomedicine, in particular to gene editing drugs directed to Chinese RHO-adRP patients and based on CRISPR/Cas9 and AAV technologies.

### BACKGROUND

Currently, retinitis pigmentosa (RP) is a group of hereditary blinding eye diseases involving the progressive losses of photoreceptor cell and/or retinal pigment epithelium cell functions as main changes. There is currently no effective therapy (traditional drugs and surgical treatments are largely ineffective). In recent years, the rapid development of CRISPR/Cas9 technology has brought a dawn for the gene therapy of RP. CRISPR/Cas9 technology is convenient and simple to operate. It is the most commonly used gene editing technology currently, and also one of the main tools for gene therapy of hereditary retinal degeneration. RHO is the first RP causative gene discovered and about 30%-40% of autosomal dominant RP (adRP) is caused by this gene, which is the most important causative gene for adRP.

Usually, RHO-adRP patients carry the causative mutation in one allele while the other allele is normal; the pathogenic mechanism of the RHO mutation is either a gain-of-function mechanism or a dominant-negative effect mechanism. It has been reported in literatures that the RHO gene p.Pro23His is a hotspot for the mutation in the RHO-adRP population in North America. At present, the gene editing therapy for RHO based on CRISPR/Cas9 technology is mostly related to this locus. However, there are few studies on RHO mutation hotspots in Asian populations, which cannot be used for gene editing therapy in Chinese RHO-adRP populations. Thus, it is necessary to study the gene mutation hotspots in Chinese populations for drug design.

The adeno-associated virus (AAV) vector as a gene transfer vector has the advantages such as non-pathogenicity, low immunogenicity, effective transfer of target gene, and long-term expression of carried therapeutic gene, and thus has become the most widely used vector for retinal gene therapy. However, the maximum carrying capacity of the AAV vector is 4.7 kb. Usually, sgRNA and Cas9 can only be packaged separately. Therefore, in order to improve the targeting efficiency, it is necessary to select a suitable vector.

### SUMMARY

The present application provides gene editing drugs directed to Chinese RHO-adRP patients based on CRISPR/Cas9 and AAV technologies.

In the present application, a mutant allele-specific gRNA is designed for the mutation hotspots of Chinese RHO-adRP populations to knock out the mutant allele while retain the normal allele so as to achieve the therapeutic purpose. Since the RHO gene mutations that cause adRP are mostly missense mutations (there is only one base difference between the mutant allele and the normal allele), when a gRNA is designed, the mutation site can be located in the gRNA, so that the gRNA is completely matched with the mutation allele while has one base difference from the normal allele. Such a gRNA is a mutant allele-specific gRNA. In certain embodiments, the present application can use the CRISPR/SaCas9 system for gene editing, wherein the SaCas9 protein and gRNA can be packaged into a single AAV virus. For example, the pX601-SaCas9 plasmid vector can be used and the gRNA and SaCas9 are packaged into an AAV vector (e.g., AAV8 vector) to improve the targeting efficiency. In certain embodiments, the vector can be injected into the eyeballs of RHO-adRP patient by subretinal injection to achieve the therapeutic purpose. The methods and compositions described in the present application can specifically cleave the relevant mutation sites of the RHO gene, have a certain cleavage efficiency and safety, have been verified in cell, tissue and animal models, and have a great application value.

In one aspect, the subject application provides a method for treating retinitis pigmentosa, comprising the step of: enabling a subject in need thereof to have a functional RHO gene, wherein the functional RHO gene does not comprise a mutation site selected from the group consisting of c.C50T and c.C403T.

In certain embodiments, the method comprises the step of: removing the mutation site of the RHO gene in the subject in need thereof.

In certain embodiments, the removing comprises knocking out the mutation site and/or reducing the expression level of the RHO gene comprising the mutation site.

In certain embodiments, the removing comprises not affecting the expression level and/or function of the wild-type RHO gene in the subject.

In certain embodiments, the removing comprises realizing a double-strand break in the RHO allele comprising the mutation.

In certain embodiments, the removing comprises administrating to the subject in need thereof at least one vector capable of removing the mutation site.

In certain embodiments, the vector comprises a sequence encoding a gRNA that specifically binds to the mutation site.

In certain embodiments, the gRNA specifically binds to at least a portion of nucleic acid in the RHO allele comprising the mutation site.

In certain embodiments, the gRNA is specifically complementary to at least a portion of nucleic acid sequence in exon 1 of the RHO allele comprising c.C50T mutation.

In certain embodiments, the gRNA specifically complementary to at least a portion of nucleic acid sequence in exon 1 of the RHO allele comprising c.C50T mutation comprises an amino acid sequence set forth in any of SEQ ID NOs: 44-45.

In certain embodiments, the sequence encoding the gRNA specifically complementary to at least a portion of nucleic acid sequence in exon 1 of the RHO allele comprising c.C50T mutation comprises a nucleotide sequence set forth in any of SEQ ID NOs. 1-2.

In certain embodiments, the gRNA is specifically complementary to at least a portion of nucleic acid sequence in exon 2 of the RHO allele comprising c.C403T mutation.

In certain embodiments, the gRNA specifically complementary to at least a portion of nucleic acid sequence in exon 2 of the RHO allele comprising c.C403T mutation comprises an amino acid sequence set forth in SEQ ID NO: 47.

In certain embodiments, the sequence encoding the gRNA specifically complementary to at least a portion of nucleic acid sequence in exon 2 of the RHO allele comprising c.C403T mutation comprises a nucleotide sequence set forth in SEQ ID NO. 4.

In certain embodiments, the vector includes a nucleic acid encoding Cas protein.

In certain embodiments, the Cas protein includes Cas9 protein.

In certain embodiments, the sequence encoding the gRNA and the nucleic acid encoding Cas protein are located in a same vector.

In certain embodiments, the vector comprises a viral vector.

In certain embodiments, the vector is an adeno-associated virus vector (AAV). In certain embodiments, the vector is AAV8.

In certain embodiments, the subject comprises East Asians.

In certain embodiments, the method is performed in vitro, in vivo, or ex vivo.

In certain embodiments, the administrating comprises injection.

In certain embodiments, the administrating comprises injection in the subretinal space.

In another aspect, the subject application provides a method for editing the RHO gene, comprising the step of: removing a mutation site selected from the group consisting of c.C50T and c.C403T in the RHO gene.

In certain embodiments, the removing comprises knocking out the mutation site and/or reducing the expression level of the RHO gene comprising the mutation site.

In certain embodiments, the removing comprises not affecting the expression level and/or function of the wild-type RHO gene in the subject.

In certain embodiments, the removing comprises realizing a double-strand break in the RHO allele comprising the mutation.

In certain embodiments, the removing comprises administrating at least one vector capable of removing the mutation site.

In certain embodiments, the vector comprises a sequence encoding a gRNA that specifically binds to the mutation site.

In certain embodiments, the gRNA specifically binds to at least a portion of nucleic acid in the RHO allele comprising the mutation site.

In certain embodiments, the gRNA is specifically complementary to at least a portion of nucleic acid sequence in exon 1 of the RHO allele comprising c.C50T mutation.

In certain embodiments, the sequence encoding the gRNA comprises a nucleotide sequence set forth in any of SEQ ID NOs. 1-2.

In certain embodiments, the gRNA is specifically complementary to at least a portion of nucleic acid sequence in exon 2 of the RHO allele comprising c.C403T mutation.

In certain embodiments, the sequence encoding the gRNA comprises a nucleotide sequence set forth in SEQ ID NO. 4.

In certain embodiments, the vector comprises a nucleic acid encoding Cas protein.

In certain embodiments, the Cas protein comprises Cas9 protein.

In certain embodiments, the sequence encoding the gRNA and the nucleic acid encoding Cas protein are located in a same vector.

In certain embodiments, the vector comprises a viral vector.

In certain embodiments, the vector is an adeno-associated virus vector (AAV).

In certain embodiments, the vector is AAV8.

In another aspect, the subject application provides a composition for treating retinitis pigmentosa in a subject, comprising an active ingredient that removes a mutation site in RHO gene, and a pharmaceutically acceptable carrier, wherein the mutation site is selected from the group consisting of c.C50T and c.C403T.

In certain embodiments, the active ingredient comprises a sequence encoding a gRNA that specifically binds to the mutation site.

In certain embodiments, the gRNA that specifically binds to the mutation site comprises a nucleotide sequence set forth in any of SEQ ID NOs. 44, 45, and 47.

In certain embodiments, the sequence encoding the gRNA comprises a nucleotide sequence set forth in any of SEQ ID NOs. 1, 2, and 4.

In certain embodiments, the active ingredient comprises Cas protein.

In certain embodiments, the Cas protein comprises Cas9 protein.

In certain embodiments, the sequence encoding the gRNA and the nucleic acid encoding Cas protein are located in a same vector.

In certain embodiments, the vector comprises a viral vector.

In certain embodiments, the vector is an adeno-associated virus vector (AAV).

In certain embodiments, the vector is AAV8.

Other aspects and advantages of the present application can be readily appreciated by those skilled in the art from the detailed descriptions below. Only exemplary embodiments of the present application are shown and described in the detailed descriptions below. As recognized by those skilled in the art, the disclosure of the present application will enable those skilled in the art to make changes to the specific embodiments without departing from the spirit and scope of the invention disclosed in the present application. Accordingly, the accompanying drawings and the descriptions in the specification of the present application are only exemplary and not limitative in any way.

### DESCRIPTION OF THE DRAWINGS

The specific features of the invention disclosed in the present application are set forth in the appended claims. The characteristics and advantages of the invention disclosed in the present application can be better understood by reference to the exemplary embodiments described in detail below and the accompanying drawings. Brief descriptions of the accompanying drawings are as follows:

FIG. 1 shows a structural characteristic map of the gene editing vectors used in the present application.

FIG. 2 shows three sequences encoding gRNAs designed for RHO p.Thr17Met.

FIG. 3 shows two sequences encoding gRNAs designed for RHO p.Arg135Trp.

FIG. 4 shows the results of in vitro editing efficiency assay of RHO17-SgRNA.

FIG. 5 shows the results of in vitro editing efficiency assay of RHO135-SgRNA.

FIG. 6 shows the results of in vitro assay of RHO17-SgRNA1 editing using the gRNA activity fluorescence assay kit.

FIG. 7 shows the results of in vitro assay of RHO17-SgRNA2 editing using the gRNA activity fluorescence assay kit.

FIG. 8 shows the results of in vitro assay of RHO17-SgRNA3 editing using the gRNA activity fluorescence assay kit.

FIG. 9 shows the results of in vitro assay of RHO135-SgRNA1 editing using the gRNA activity fluorescence assay kit.

FIG. 10 shows the results of in vitro assay of RHO135-SgRNA2 editing using the gRNA activity fluorescence assay kit.

FIG. 11 shows the statistics of flow sorting results of in vitro assay of sgRNA editing efficiency using the gRNA activity fluorescence assay kit.

FIG. 12 shows the results of in vitro verification of sgRNA safety and specificity in 293T cells by T7E1 detection.

FIG. 13 shows the gene editing efficiency of RHO17-SgRNA1 and RHO17-SgRNA2 only in patient iPSCs with T17M mutation.

FIG. 14 shows the gene editing efficiency of RHO135-SgRNA1 only in patient iPSCs with R135W mutation.

FIG. 15 shows the gene editing effect of RHO17-SgRNA2 and RHO135-SgRNA1 on the 3D retinal organoid of patient.

FIG. 16 shows the genotype identification results of RHO humanized mice.

FIG. 17 shows the gene editing effect of RHO17-SgRNA2 and RHO135-SgRNA1 on the retinal tissue of humanized mouse.

### DESCRIPTION OF EMBODIMENTS

The embodiments of the invention in the present application are described below by certain specific examples, and those skilled in the art can easily understand other advantages and effects of the invention in the present application from the disclosure of this specification.

### Definitions of Terms

In the present application, the term “c.C50T” generally refers to the base mutation from cytosine (C) to thymine (T) at position 50 of the coding sequence of the RHO gene (from the 5′ end to the 3′ end, “A” in the initiation ATG of the coding sequence as position 1) as compared to the nucleotide sequence of the wild-type RHO gene. In the present application, “c.” generally refers to the coding sequence, which means a sequence from the initiation codon ATG to the termination codon, and the coding sequence can initiate and terminate at any position in the mRNA. Thus, “c.C50T” represents that starting from A of the ATG in the coding sequence, the 50th nucleotide is mutated from C to T. Such a base mutation can lead to the change of the amino acid encoded by the RHO gene; for example, the amino acid is mutated from threonine (Thr) to methionine (Met). In the present application, the term “p.Thr17Met” generally refers to the mutation of the amino acid at position 17 of RHO protein from threonine (Thr) to methionine (Met).

In the present application, the term “c.C403T” generally refers to the base mutation from cytosine (C) to thymine (T) at position 403 of the coding sequence of the RHO gene (from the 5′ end to the 3′ end, “A” in the initiation ATG of the coding sequence as position 1) as compared to the nucleotide sequence of the wild-type RHO gene. Such a base mutation can lead to the change of the amino acid encoded by the RHO gene; for example, the amino acid is mutated from arginine (Arg) to tryptophan (Trp). In the present application, the term “p.Arg135Trp” generally refers to the mutation of the amino acid at position 135 of RHO protein from arginine (Arg) to tryptophan (Trp).

In the present application, the term “exon 1 of the RHO allele” generally refers to the first exon in the RHO gene. For example, the exon 1 of the RHO allele has an ID of ENSE00001079597 in the Ensembl database, which may include the nucleotide sequence of positions 129,528,639-129,529,094 of Homo sapiens chromosome 3.

In the present application, the term “exon 5 of the RHO allele” generally refers to the fifth exon in the RHO gene. For example, the exon 5 of the RHO allele has an ID of ENSE00001079599 in the Ensembl database, which may include the nucleotide sequence of positions 129,533,608-129,535,344 of Homo sapiens chromosome 3.

In the present application, the term “double-strand break (DSB)” generally refers to a phenomenon occurred when two single strands of a double-stranded DNA molecule are cleaved at the same position. The double-strand break can induce a DNA repair, possibly causing the genetic recombination. The cells also have certain systems that act on the double-strand breaks generated at other times. The double-strand break can periodically occur during the normal cell replication cycle or can be enhanced in some cases, such as by UV light and DNA break inducer (e.g., various chemical inducers). Many inducers can lead to the indiscriminate occurrence of DSB across the genome, and the DSB can be regularly induced and repaired in normal cells. During the repair, the original sequence can be reconstructed with full fidelity, but in some cases, small insertions or deletions (called as “indels”) may be introduced at DSB sites. In certain instances, the double-strand break can also be specifically induced at a specific position, which may be used to cause a targeted or preferential genetic modification at a selected chromosomal position. In many instances, the tendency of recombination for homologous sequences during the DNA repair (and replication) can be exploited, which underlies the application of a gene editing system (such as CRISPR). This homology-directed repair is used to insert the sequence of interest provided by the use of a “donor” polynucleotide into the desired chromosomal position.

The term “knock out” refers to an alteration in the nucleic acid sequence of a gene that reduces the biological activity of the polypeptide typically encoded by the gene by at least 80% as compared to the unaltered gene. For example, the alteration can be an insertion, substitution, deletion, frame-shift mutation, or missense mutation of one or more nucleotides.

In the present application, the term “complementary” generally refers that a nucleic acid (e.g., RNA) comprising a nucleotide sequence that enables the nucleic acid to bind non-covalently (e.g., by Watson-Crick base pairing) “hybridizes” or “complements” to another nucleic acid in a sequence-specific, anti-parallel manner (i.e., nucleic acid specifically binds to a complementary nucleic acid) under appropriate in vitro and/or in vivo conditions of temperature and solution ionic strength. As known in the art, the standard Watson-Crick base pairing includes: pairing between adenine (A) and thymidine (T), pairing between adenine (A) and uracil (U), and pairing between guanine (G) and cytosine (C).

As used herein, the terms “polypeptide,” “peptide,” and “protein” are used interchangeably, and generally refer to a polymer of amino acids of any length. The polymer may be linear or branched, and it may contain modified amino acids, and may be interrupted by non-amino acids. These terms also encompass amino acid polymers that have been modified. These modifications may include: disulfide bond formation, glycosylation, lipidation, acetylation, phosphorylation, or any other manipulation (e.g., binding to a labeling component). The term “amino acid” includes natural and/or non-natural or synthetic amino acids, including glycine and D and L optical isomers, as well as amino acid analogs and peptidomimetics.

The terms “polynucleotide,” “nucleotide,” “nucleotide sequence,” “nucleic acid,” and “oligonucleotide” are used interchangeably, and generally refer to a polymeric form of nucleotides (such as deoxyribonucleotides or ribonucleotides) of any length, or analogs thereof. The polynucleotide can have any three-dimensional structure and can perform any known or unknown function. The non-limiting examples of the polynucleotide are as follows: coding or non-coding region of gene or gene fragment, multiple loci (one locus) defined by ligation analysis, exon, intron, messenger RNA (mRNA), transporter RNA, ribosomal RNA, short interfering RNA (siRNA), short hairpin RNA (shRNA), micro-RNA (miRNA), ribozyme, cDNA, recombinant polynucleotide, branched polynucleotide, plasmid, vector, isolated DNA of any sequence, isolated RNA of any sequence, nucleic acid probe, and primer. The polynucleotide may contain one or more modified nucleotides, such as methylated nucleotides and nucleotide analogs. If present, the modification of nucleotide structure can be performed before or after polymer assembly. The sequence of the nucleotide can be interrupted by a non-nucleotide component. The polynucleotide can be further modified after polymerization, such as by conjugation to a labeling component.

In the present application, the “vector” generally refers to a nucleic acid molecule capable of self-replication in a suitable host for transferring the inserted nucleic acid molecule into and/or among host cells. The vector may include vectors primarily used for insertion of DNA or RNA into cells, vectors primarily used for replication of DNA or RNA, and vectors primarily used for expression of transcription and/or translation of DNA or RNA. The vector also includes a vector having a number of the above-mentioned functions. The vector may be a polynucleotide capable of being transcribed and translated into a polypeptide when introduced into a suitable host cell. Typically, the vector can produce the desired expression product by culturing a suitable host cell containing the vector.

In the present application, the term “plasmid” generally refers to DNA molecules other than chromosomes or nucleoids in organisms such as bacteria and yeast, which exist in the cytoplasm and have the ability to autonomously replicate, enable to maintain a constant copy number in progeny cells, and express the carried genetic information. The plasmid is used as the vector for genes in the genetic engineering research.

In the present application, the term “retroviral vector” generally refers to a virus particle that can be controllable and express foreign genes, but cannot be self-packaged into a proliferative virus particle. Most of such viruses have reverse transcriptase. The retrovirus contains at least three genes: gag, that contains the genes for the proteins constituting the center and structure of the virus; pol, that contains the gene for reverse transcriptase; and env, that contains the gene constituting the viral coat. Through retroviral transfection, the retroviral vector can randomly and stably integrate its own genome and the carried foreign genes into the host cell genome; for example, a CAR molecule can be integrated into the host cell.

In the present application, the term “lentiviral vector” generally refers to a diploid RNA viral vector that is a retrovirus. The lentiviral vector is based on the genome of the lentivirus, and many sequence structures related to viral activity are removed to make it biologically safe, and then the sequence and expression structure of the target gene required for the experiment is introduced into this genome backbone to prepare the vector. Through transfection by lentiviral vector, the retroviral vector can randomly and stably integrate its own genome and the carried foreign genes into the host cell genome; for example, a CAR molecule can be integrated into the host cell.

In the present application, the term “and/or” should be understood to mean either or both of the options.

As used herein, the term “comprise/include” generally means the inclusion of expressly specified features, but without the exclusion of other elements.

As used herein, the term “about” generally refers to variations above or below the specified value within the range of 0.5%-10%, such as variations above or below the specified value within the range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%.

### DETAILED DESCRIPTION OF THE INVENTION

RP and RHO Genes

In one aspect, the present application provides a method of treating retinitis pigmentosa. In the present application, the term “retinitis pigmentosa (RP)” generally refers to a genetic disease that causes the retinal degeneration. More than 80 RP-related genes have been identified, and these genes are involved in autosomal recessive inheritance (50-60%), autosomal dominant inheritance (AD, 30-40%), and X-linked inheritance (5-15%). RP is characterized by the progressive vision loss due to the dysfunction of the photoreceptor cells (cones and rods) and/or retinal pigment epithelium cells. The clinical manifestations of RP may include night blindness, progressive visual field defect, central vision diminution after macular involvement, and eventual blindness. The electroretinogram (ERG) shows that the function of rods is decreased or even extinguished. The main fundus change of RP is retinal pigment disorder in the equatorial region, with osteocytic pigmentation, which gradually develops towards the posterior pole and the ora serrata. RPEs, photoreceptor cells, and laminae choriocapillaris are gradually atrophied, the large choroidal vessels are thoroughly seen, the retina is blue-gray, the retinal arteries are thinned, and the optic disc is sallow and atrophied. Among them, retinal vascular stenosis, sallow optic disc, and osteocytic pigmentation are typical triad of RP (Hartong D T et al., 2006). The methods for assessing the retinal function and morphology may include best corrected visual acuity (BCVA), fundus autofluorescence, perimetry, ERG, fundus photograph, optical coherence tomography (OCT), fluorescein angiography (FFA), and the like. The methods for assessing the visual function may include BCVA, perimetry, and the like.

The method described herein may include imparting a functional RHO gene to a subject in need thereof.

In certain instances, the RP described herein may be caused by the mutation(s) in the RHO gene. There are many RHO gene mutations associated with RP, and these gene mutations can cause the RHO gene to encode abnormally functional rhodopsin. The mutations may include but not limited to the missense, nonsense, insertion, deletion, and other mutations of the gene. For example, the mutation site may comprise a mutation site selected from the group consisting of c.C50T and c.C403T. Again, for example, the mutation site may result in an amino acid change, and the amino acid mutation may comprise the following changes: p.Thr17Met and/or p.Arg135Trp. In the present application, the method may include depriving a subject in need thereof of the RHO gene with a heterozygous mutation site, wherein the mutation site may be selected from the group consisting of c.C50T and c.C403T.

Any one or more mutations may be repaired to impart a functional RHO gene to a subject in need thereof. For example, c.C50T and/or c.C403T as pathological variants can be removed, restored, or corrected.

In the present application, the term “functional RHO gene” generally refers to a gene capable of encoding the rhodopsin with normal functions. In the present application, this term refers to a RHO gene without a mutation site. For example, the mutation site may be selected from the group consisting of c.C50T and c.C403T. In certain instances, the RHO gene containing mutation sites (e.g., c.C50T and c.C403T) may be turned into a functional RHO gene. For example, the mutation sites (e.g., c.C50T and c.C403T) can be specifically removed. Again, for example, the mutation sites (e.g., c.C50T and c.C403T) may be removed by gene knockout in combination with homology directed repair (HDR), and the RHO gene containing mutation sites (e.g., c.C50T and c.C403T) is turned into a functional RHO gene.

The term “RHO gene” may also be referred to as Rhodopsin 2, Opsin-2, Opsin 2, OPN2, CSNBAD1, or RP4. The rhodopsin is located in the rods outer segment (ROS), and is necessary for normal vision, especially for the perception of weak light stimuli. The rods are a kind of retinal photoreceptor cells for scotopic vision. The cones are another kind of retinal photoreceptor cells for photopic vision and color vision. At the ROS, the rhodopsin usually binds to 11-cis retinal (11cRAL) which is a derivative form of vitamin A. The absorption of photons by ROS turns the rhodopsin into the active rhodopsin (R*), and isomerizes 11cRAL to all-trans retinal (atRAL). The atRAL is quickly reduced to all-trans retinol (atROL) after separation from R*. The interphotoreceptor retinoid-binding protein (IRBP) is responsible for transferring the atROL into RPE cells, where the atROL is converted by the lecithin retinol acyltransferase (LRAT) to all-trans retinol ester, which is further converted to 11-cis retinol ester and then isomerized to 11-cis retinol (11cROL) by the hydrolytic isomerase RPE65. The 11cROL is oxidized by RDHs to 11cRAL, which binds to IRBP and is transported back to photoreceptor cells for reuse. R* converts the GDP on the a subunit of transducin G (Gt) in the downstream membranous disc into GTP, such that the a subunit is separated from the βγ subunit, the cyclic guanosine monophosphate-phosphodiesterase 6 (cGMP-PDE6) is activated, and the cGMP is hydrolyzed. The concentration of cellular cGMP is reduced to close the cGMP-gated cation channel of OS, such that the Ca²⁺ concentration within photoreceptor cells is reduced, the cell membrane is hyperpolarized, and the light signal is converted into a visual electrical signal. After the phototransduction is terminated, the photoreceptor cells are subjected to a series of chemical reactions and return to the non-illuminated state. At this time, R* is phosphorylated and binds to the arrestin to inhibit the downstream signaling pathway. The PDE6 is in an inactive state, while the cGMP is synthesized in the rods. The increased cGMP concentration makes the cation channel open to achieve Ca²⁺ influx, and the cell membrane is depolarized. Such nerve impulses triggered by the opening and closing of cGMP-gated cation channels are transmitted to the visual center of the cerebral cortex through the connections between the synaptic terminals of photoreceptor cells and the neurons at all levels in the retina as well as the optic nerve to form the vision.

The human RHO gene is located at position 22.1 of the long arm of chromosome 3 (3q22.1), and the molecule is from 129,528,639 bp to 129,535,344 bp in chromosome 3 (Homo sapiens, Annotation release, Version 109.20200228, GRCh38.p13, NCBI). The nucleotide sequence of the RHO gene can be found in NCBI GenBank Accession No. NG_009115.1. The RHO gene has 5 exons. Table 1 shows the exon identifiers of the RHO gene and the exon initiation/termination sites in the Ensembl database.

TABLE 1
Exons of the RHO gene

| | Exon ID | Initiation/termination site (chromosome 3) |
|---|---|---|
| 1 | ENSE00001079597 | 129,528,639-129,529,094 |
| 2 | ENSE00001152211 | 129,530,876-129,531,044 |
| 3 | ENSE00001152205 | 129,532,251-129,532,416 |
| 4 | ENSE00001152199 | 129,532,533-129,532,772 |
| 5 | ENSE00001079599 | 129,533,608-129,535,344 |

The RHO gene disfunction may be due to gene mutations, including but not limited to the insertion, deletion, missense, nonsense, frame-shift, and/or other mutations of the nucleotide. In certain instances, any one or more mutations may be repaired to restore the normal functions of the RHO gene. For example, the mutation sites in the RHO gene may be removed.

In certain instances, the method may comprise the step of: removing the mutation site of the RHO gene in the subject in need thereof. The method may include exon deletion. The targeted deletion of specific exon may be a strategy for treating a large number of patients with a single therapeutic cocktail therapy. The exon deletion may be single-exon deletion or multi-exon deletion. Although the multi-exon deletion may cover more subjects, for deletions of more nucleotides, the efficiency of the deletion greatly decreases with the increased nucleotide size. Thus, the removal can range from 40 to 10,000 base pairs (bp). For example, the removal can range from 40-100, 100-300, 300-500, 500-1,000, 1,000-2,000, 2,000-3,000, 3,000-5,000, or 5,000-10,000 base pairs.

As above mentioned, the RHO gene contains 5 exons. Any base or bases in the 5 exons can contain the mutation(s). Any one or more of the mutation sites in five mutated exons or abnormal intron splice acceptor or donor sites can be removed so that the functional RHO gene excludes the mutation site (e.g., the mutation affecting the function of the RHO gene). In certain instances, the mutation site may be from any one or more of the following group of the RHO gene: exon 1, exon 2, exon 3, exon 4, exon 5, or any combination thereof. For example, the gene mutation may be a mutation selected from the group consisting of c.C50T and c.C403T. The gene mutation can lead to amino acid mutation, and eventually lead to the abnormal function of RHO protein. For example, the mutant protein can interfere with the function of normal protein or cannot be located to the ROS.

In Vivo or In Vitro Method

The method described herein may comprise knocking out the mutation site and/or reducing the expression level of the mutation site. The methods for knocking out a gene or reducing the expression level of a gene may include gene knockout, conditional gene knockout (e.g., using the Cre/LoxP and/or FLP-frt system), inducible gene knockout (e.g., knockout based on the Cre/loxp system, including tetracycline-induced, interferon-induced, hormone-induced, and adenovirus-induced gene knockout, etc.), gene knockout by random insertion mutation (e.g., gene trapping method), gene knockout by RNAi, gene editing technology mediated by zinc finger nucleases (ZNF), gene editing technology mediated by transcription activator-like effector nucleases (TALEN), gene editing technology mediated by clustered regularly interspaced short palindromic repeats (CRISPR)/CRISPR associated proteins (Cas) (CRISPR/Cas) system, and/or NgAgo-gDNA gene editing technology. For any genome editing strategy, the gene editing may be confirmed by sequencing or PCR analysis.

In certain instances, the method described herein may be a method based on in vivo cells. In certain instances, the method comprises editing the genomic DNA of the subject's cells. For example, the method may include editing the mutation in the RHO gene in the subject's cells (e.g., photoreceptor cells and/or retinal progenitor cells). For example, the gene mutation may be a mutation selected from the group consisting of c.C50T and c.C403T. Although certain cells may be ideal targets for ex vivo methods or therapies, the use of effective delivery method may also allow for the direct delivery in vivo of desired agents to such cells. In certain instances, the method may include targeting and editing relevant cells. The lysis of other cells can also be prevented by using the promoters that are active only in certain cells and/or developmental stages.

The added promoter is inducible; thus, if the nucleic acid molecule is delivered in a plasmid vector, the timing of delivery can be controlled. The period for which the delivered nucleic acid or protein remains within the cell can also be adjusted by altering the half-life. The in vivo method can save some processing steps, but require a higher editing efficiency. The in vivo treatment can eliminate the problems and losses with ex vivo treatment and implantation.

The in vivo method can facilitate the production and administration of the therapeutic product. The same treatment or therapy will potentially be used to treat more than one subject, for example, many subjects with the same or similar genotypes or alleles.

The method described herein may include an ex vivo method. In certain instances, the subject-specific induced pluripotent stem cells (iPSCs) may be obtained. Then, the genomic DNA of these iPSC cells may be edited using the method described herein. For example, this method may include editing at or near the mutation site of the RHO gene of the iPSC, such that it does not have the amino acid mutations of p.Thr17Met and/or p.Arg135Trp. For example, the gene mutation may be a mutation selected from the group consisting of c.C50T and c.C403T. Next, the gene-edited iPSCs may be differentiated into other cells, such as photoreceptor cells or retinal progenitor cells. Finally, the differentiated cells (e.g., photoreceptor cells or retinal progenitor cells) may be implanted into the subject.

In other instances, the photoreceptor cells or retinal progenitor cells may be isolated from the subject. Next, the genomic DNA of these photoreceptor cells or retinal progenitor cells may be edited using the method described herein. For example, this method may include editing at or near the mutation site of the RHO gene of the photoreceptor cells or retinal progenitor cells, such that it does not have the amino acid mutations of p.Thr17Met and/or p.Arg135Trp. For example, the gene mutation may be a mutation selected from the group consisting of c.C50T and c.C403T. Finally, the gene-edited photoreceptor cells or retinal progenitor cells may be implanted into the subject.

In other instances, the mesenchymal stem cells may be isolated in vivo, or in other instances isolated from the bone marrow or peripheral blood. Next, the genomic DNA of these mesenchymal stem cells may be edited using the method described herein. For example, this method may include editing at or near the mutation site of the RHO gene of the mesenchymal stem cells, such that it does not have the amino acid mutations of p.Thr17Met and/or p.Arg135Trp. For example, the gene mutation may be a mutation selected from the group consisting of c.C50T and c.C403T. Next, the gene-edited mesenchymal stem cells may be differentiated into any type of cells, such as photoreceptor cells or retinal progenitor cells. Finally, the differentiated cells such as photoreceptor cells or retinal progenitor cells may be implanted into the subject.

The method may include a comprehensive analysis of the therapeutic agent prior to administration. For example, the entire genome of the corrected cell is sequenced to ensure that no off-target effects, if any, can be at the genomic positions associated with minimal risk to the subject. Moreover, a population of specific cell (including clonal cell population) may be isolated prior to implantation.

The use of the method described herein may not affect the expression level and/or function of the wild-type RHO gene in the subject.

Gene Editing

The method described herein may include the process for cleaving DNA at a precise target position in the genome using a site-directed nuclease, thereby producing single- or double-stranded DNA breaks at the specific position within the genome. Such breaks may be periodically repaired by endogenous cellular processes such as HDR and non-homologous end joining (NHEJ). These two major DNA repair processes consist of a series of alternative pathways. NHEJ directly joins the ends of DNA resulting from double-strand breaks, sometimes missing or adding nucleotide sequences, which may disrupt or enhance the gene expression. HDR uses the homologous or donor sequence as template to insert a specific DNA sequence at the breakpoint. The homologous sequence may be in the endogenous genome, such as sister chromatid. Alternatively, the donor may be an exogenous nucleic acid, such as plasmid, single-stranded oligonucleotide, double-stranded oligonucleotide, or virus. These exogenous nucleic acids may contain regions of high homology to the nuclease-cleavable locus, and may also contain additional sequences or sequence changes (including deletions that can be incorporated into the cleavable target locus). A third repair mechanism can be the microhomology-mediated end joining (MMEJ), also known as “alternative NHEJ (ANHEJ)”, where small deletions and insertions may occur at the cleavage site, and which genetic results are similar to NHEJ. MMEJ can utilize the homologous sequences of several base pairs flanking the DNA break site to drive more favorable repair outcomes of DNA end-joining. In certain instances, it may be possible to predict the possible repair outcomes based on the analysis of potential microscopic homology at the DNA break site.

All of these gene editing mechanisms can be used to remove the gene mutation sites required in the present application. The methods described herein may include creating one DNA break, or two DNA breaks which may be double-stranded breaks or two single-stranded breaks, at the position near the site of the intended mutation in the target locus. In certain instances, the removing may include realizing a double-strand break in the RHO allele comprising the mutation. The break may be achieved by a site-directed polypeptide. The site-directed polypeptide (e.g., DNA endonuclease) can introduce double- or single-stranded breaks in the nucleic acid (e.g., genomic DNA). The double-stranded break can stimulate the endogenous DNA repair pathway in a cell, such as HDR, NHEJ, or MMEJ. NHEJ can repair the cleaved target nucleic acid without the homologous template.

In certain instances, the homologous recombination may be used to insert an exogenous polynucleotide sequence into the target nucleic acid cleavage site. The exogenous polynucleotide sequence may be referred to as a donor polynucleotide (or donor, or donor sequence, or polynucleotide donor template). The donor polynucleotide, a portion of the donor polynucleotide, a copy of the donor polynucleotide, or a portion of a copy of the donor polynucleotide may be inserted into the target nucleic acid cleavage site. The donor polynucleotide may be an exogenous polynucleotide sequence, that is, a sequence that is not naturally present at the target nucleic acid cleavage site.

HDR occurs when a homologous repair template or donor is available. The homologous donor template may comprise at least a portion of the wild-type RHO gene or cDNA. At least a portion of the wild-type RHO gene or cDNA may be exon 1, exon 2, exon 3, exon 4, exon 5, intron regions, fragments or combinations of the above, or the entire RHO gene or cDNA. The donor template may be a single-stranded or double-stranded polynucleotide. The donor template may be delivered by AAV. The homologous donor template may comprise sequences homologous to the sequences flanking the target nucleic acid cleavage site. For example, the donor template may have arms homologous to the 3q22.1 region. The donor template may also have arms homologous to pathological variants c.C50T and/or c.C403T. The sister chromatid may be used as repair template by the cell. However, for the purpose of gene editing, the repair template may be provided as exogenous nucleic acid, such as plasmid, double-stranded oligonucleotide, single-stranded oligonucleotide, or viral nucleic acid. Using an exogenous donor template, an additional nucleic acid sequence (e.g., transgene) or modification (e.g., single or multiple base changes or deletions) may be introduced among homologous flanking regions, and thus an additional or altered nucleic acid sequence may be incorporated into the target locus. MMEJ can utilize the homologous sequences of several base pairs flanking the cleavage site to drive favorable repair outcomes of DNA end-joining. In certain instances, it may be possible to predict the possible repair outcomes based on the analysis of potential microscopic homology in the nuclease-targeted region.

CRISPR/Cas System

In the present application, the term “CRISPR/Cas system” or “CRISPR-Cas system” generally refers to a nuclease system consisting of clustered regularly interspaced short palindromic repeats (CRISPR) and CRISPR associated proteins (i.e., Cas proteins), which is capable of cleaving almost all genomic sequences adjacent to the protospacer-adjacent motif (PAM) in eukaryotic cells. The “CRISPR/Cas system” may be used to collectively refer to transcripts involving CRISPR-associated (“Cas”) genes, as well as other elements involved in their expressions thereof or guiding their activities, and may include the sequence encoding a Cas gene, tracr (transactivating CRISPR) sequence (e.g., tracrRNA or active part thereof), tracr partner sequence (encompassing “direct repeats” and processed partial direct repeats, in the context of endogenous CRISPR/Cas system), guide sequence (also known as “spacer” in the context of endogenous CRISPR/Cas system), or other sequences and transcripts from CRISPR loci. Five types of CRISPR systems have been identified (e.g., Type I, Type II, Type III, Type U, and Type V).

In the present application, the term “Cas protein”, also known as “CRISPR-associated protein”, generally refers to a class of enzymes that are complementary to CRISPR sequences and can use the CRISPR sequences as guides to recognize and cleave specific DNA strands. The non-limiting examples of Cas proteins include: Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and/or their homologs, or modified forms thereof. In some embodiments, the Cas protein is a Cas9 protein.

In the present application, the term “Cas9 protein” or “Cas9 nuclease”, also known as Csn1 or Csx12, generally refers to a class of proteins that are involved in both crRNA biosynthesis and destruction of invading DNA in the Type II CRISPR/Cas system. The Cas9 protein typically includes a RuvC nuclease domain and an HNH nuclease domain, which cleave two different strands of a double-stranded DNA molecule, respectively. The Cas9 protein has been described in different bacterial species such as S. thermophiles, Listeria innocua (Gasiunas, Barrangou et al. 2012; Jinek, Chylinski et al. 2012), and (S. Pyogenes) (Deltcheva, Chylinski et al. 2011). For example, the amino acid sequence for Streptococcus pyogenes Cas9 protein can be found in SwissProt database with accession number Q99ZW2; the amino acid sequence for Neisseria meningitides can be found in UniProt database with number A1IQ68; the amino acid sequence for Streptococcus thermophilus can be found in UniProt database with number Q03LF7; and the amino acid sequence for Staphylococcus aureus can be found in UniProt database with number J7RUA5.

The CRISPR/Cas system may comprise many short repetitive sequences, called as “repeats.” When expressed, the repetitive sequences can form secondary structures (e.g., hairpins) and/or comprise unstructured single-stranded sequences. The repetitive sequences usually occur in clusters and often diverge among species due to evolutions. These repetitive sequences are regularly spaced with unique intermediate sequences called “spacers”, thereby forming a repeat-spacer-repeat locus structure. The spacers are identical or highly homologous to known foreign invader sequences. The spacer-repeat unit encodes crispRNA (crRNA), which is processed into a mature form of the spacer-repeat unit. The crRNA contains a “seed” or spacer sequence (naturally occurring form in prokaryotes, and the spacer sequence targets foreign invader nucleic acids) that targets the target nucleic acid. The spacer region sequence is located at the 5′ or 3′ end of the crRNA.

The CRISPR/Cas system may also comprise a polynucleotide sequence encoding the CRISPR-associated protein (Cas protein). The Cas gene encodes a nuclease involved in the biogenesis and interference stages of crRNA function in the prokaryote. Some Cas genes contain homologous secondary and/or tertiary structures.

In nature, the crRNA biosynthesis in the Type II CRISPR system requires transactivating CRISPR RNA (tracrRNA). The tracrRNA may be modified by endogenous RNaseIII and then hybridize to crRNA repeats in the pre-crRNA. The endogenous RNaseIII may be recruited to cleave the pre-crRNA. The cleaved crRNA may be trimmed by exonuclease to generate the mature crRNA form (e.g., trimmed at the 5′ end). The tracrRNA may remain hybridized to crRNA, and tracrRNA and crRNA are associated with the site-directed polypeptide (e.g., Cas9). The crRNA in the crRNA-tracrRNA-Cas9 complex may direct this complex to a target nucleic acid that can hybridize to the crRNA. The hybridization of crRNA to the target nucleic acid may activate Cas9 for target nucleic acid cleavage. The target nucleic acid in the Type II CRISPR system is called as a protospacer-adjacent motif (PAM). Indeed, PAM is essential to facilitate the binding of the site-directed polypeptide (e.g., Cas9) to the target nucleic acid. The Type II system (also known as Nmeni or CASS4) can be further subdivided into Type II-A (CASS4) and Type II-B (CASS4a). A CRISPR/Cas9 system useful for RNA-programmable gene editing can be found in Jinek et al., Science, 337(6096):816-821(2012), and International Patent Application Publication No. WO2013/176772 provides numerous examples and applications of the CRISPR/Cas endonuclease system useful for site-specific gene editing.

gRNA

The method of the present application comprises providing a genome-targeted nucleic acid that can direct relevant active polypeptides (e.g., Cas protein) to the specific target sequence (e.g., RHO alleles) within the target nucleic acid. The genome-targeted nucleic acid may be RNA. Herein, the genome-targeted RNA may be referred to as “guide RNA” or “gRNA.” In certain instances, the gRNA described herein may be complementary to the target nucleic acid. In other instances, the gRNA may be identical to the target nucleic acid (when speaking of being the same, “U” in the RNA corresponds to the thymine “T” in the DNA due to the difference in the bases encoding RNA and DNA). In other instances, the nucleic acid sequence (e.g., DNA) encoding the gRNA may be the same or complementary to the target nucleic acid. In the present application, the terms “target nucleic acid,” “nucleic acid of interest,” and “target region” are used interchangeably, and usually refer to a nucleic acid sequence that can be recognized by a gRNA. The target nucleic acid may refer to a double-stranded nucleic acid or a single-stranded nucleic acid. The gRNA may be obtained by the transcription or replication from the sequence encoding it. For example, the gRNA may be obtained by the transcription from the DNA sequence encoding it. In the present application, the term “sequence encoding the gRNA” generally refers to a DNA sequence from which the gRNA can be obtained by transcription. In the present application, the “sequence encoding the gRNA” may have the same nucleotide sequence as the target sequence of the gRNA.

In certain instances, the gRNA may comprise at least a spacer sequence and a CRISPR repeat that hybridize to the target nucleic acid sequence of interest. In the Type II system, the gRNA also comprises a second RNA called as tracrRNA sequence. In the Type II CRISPR system, the CRISPR repeat and tracrRNA sequence hybridize to each other to form a duplex. The gRNA can bind to the Cas protein to form a guide RNA-Cas protein complex. The genome-targeted nucleic acid can confer the target specificity to the complex due to its association with the Cas protein. Thus, the genome-targeted nucleic acid can guide the activity of the Cas protein. In certain instances, the genome-targeted nucleic acid may be a double-stranded guide RNA. In certain instances, the gRNA may be a single-stranded guide RNA (sgRNA). The double-stranded guide RNA or single-stranded guide RNA may be modified.

In certain instances, the double-stranded guide RNA may comprise two RNA strands. The first strand may comprise an optional spacer extension sequence, a spacer sequences, and a minimal CRISPR repeat. The second strand may comprise a minimal tracrRNA sequence (complementary to the minimal CRISPR repeat), a 3′ tracrRNA sequence, and an optional tracrRNA extension sequence.

In some instances, the sgRNA may comprise, in the 5′ to 3′ direction, an optional spacer extension sequence, a spacer sequence, a minimal CRISPR repeat, a single-molecule guide linker, a minimal tracrRNA sequence, a 3′ tracrRNA sequence, and an optional tracrRNA extension sequence. The optional tracrRNA extension may contain the elements that contribute an additional functionality (e.g., stability) to the guide RNA. The single-molecule guide linker can connect the minimal CRISPR repeats and the minimal tracrRNA sequences to form a hairpin structure. The optional tracrRNA extension may contain one or more hairpins.

For example, the sgRNA may comprise a spacer sequence with variable length, having 17-30 nucleotides at the 5′ end of the sgRNA sequence. In other instances, the sgRNA may comprise a spacer sequence with variable length, having 17-24 nucleotides at the 5′ end of the sgRNA sequence. For example, the sgRNA may comprise a sequence with 21 nucleotides. For example, the sgRNA may comprise a sequence with 20 nucleotides. For example, the sgRNA may comprise a sequence with 19 nucleotides. For example, the sgRNA may comprise a sequence with 18 nucleotides. For example, the sgRNA may comprise a sequence with 17 nucleotides. For example, the sgRNA may comprise a sequence with 22 nucleotides. For example, the sgRNA may comprise a sequence with 23 nucleotides. For example, the sgRNA may comprise a sequence with 24 nucleotides. The sgRNA may be unmodified or modified.

The gRNA described herein may bind to the sequence in the target nucleic acid of interest. The genome-targeted nucleic acid (or a portion thereof) can interact with the target nucleic acid in a sequence-specific manner by hybridization (i.e., base pairing). The nucleotide sequence of the sgRNA may vary depending on the sequence of the target nucleic acid of interest.

In the CRISPR/Cas system of the present application, the gRNA sequence may be designed to hybridize to a target nucleic acid in the vicinity of a PAM sequence recognizable by the Cas protein used in the system. The gRNA may be completely matched to the target sequence, or may not be matched to the target sequence. The Cas protein usually has a specific PAM sequence recognizable in the target DNA.

For example, the Cas9 protein may be derived from S. pyogenes, and such a Cas9 protein recognizes a PAM comprising the sequence 5′-NRG-3′ in the target nucleic acid, where R comprises A or G, and N may be any nucleotide. Again, for example, the Cas9 protein may be derived from Staphylococcus aureus, and such a Cas9 protein (SaCas9) recognizes a PAM comprising the sequence 5′-NNGRR(T)-3′ in the target nucleic acid, where R comprises A or G, and N may be any nucleotide. In some more specific instances, the PAM sequence recognized by SaCas9 may comprise 5′-NNGRR-3′, where R comprises A or G, and N may be any nucleotide. For example, the PAM described herein may comprise a nucleotide sequence set forth in any of SEQ ID NOs: 39-43.

The gRNA described herein may specifically bind to the mutation site.

In certain instances, the gRNA may specifically bind to at least a portion of nucleic acid in the RHO allele comprising the mutation site. For example, the sequence encoding the gRNA may comprise a nucleotide sequence set forth in any of SEQ ID NOs. 1, 2, and 4.

In certain instances, the gRNA may be specifically complementary to at least a portion of nucleic acid sequence in exon 1 of the RHO allele comprising c.C50T mutation. For example, the gRNA specifically complementary to at least a portion of nucleic acid sequence in exon 1 of the RHO allele comprising c.C50T mutation may comprise a nucleotide sequence set forth in any of SEQ ID NOs: 44-45. For example, the sequence encoding the gRNA may comprise a nucleotide sequence set forth in any of SEQ ID NOs. 1-2.

In certain instances, the gRNA may be specifically complementary to at least a portion of nucleic acid sequence in exon 2 of the RHO allele comprising c.C403T mutation. For example, the gRNA specifically complementary to at least a portion of nucleic acid sequence in exon 2 of the RHO allele comprising c.C403T mutation may comprise a nucleotide sequence set forth in SEQ ID NO: 47. For example, the sequence encoding the gRNA may comprise a nucleotide sequence set forth in SEQ ID NO. 4.

In some instances, the complementarity percentage between the gRNA and the target nucleic acid may be at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some instances, the complementarity percentage between the gRNA and the target nucleic acid may be up to about 30%, up to about 40%, up to about 50%, up to about 60%, up to about 65%, up to about 70%, up to about 75%, up to about 80%, up to about 85%, up to about 90%, up to about 95%, up to about 97%, up to about 98%, up to about 99%, or 100%.

The gRNA described herein for use in the CRISPR system may be synthesized by chemical methods, such as high performance liquid chromatography. For example, two or more RNA molecules are linked together. The RNAs with longer lengths, such as those encoding Cas9, may be obtained by enzymatic reactions. In the art, various types of RNA modifications may be introduced during or after the chemical and/or enzymatic synthesis of RNA, such as modifications to enhance stability, reduce innate immune response, and/or enhance other properties.

Vector

The present application provides a vector. The polynucleotide (RNA or DNA) of the guide RNA and/or the polynucleotide (RNA or DNA) encoding the endonuclease may be delivered by a viral or non-viral delivery vector known in the art. Alternatively, the endonuclease polypeptide may be delivered by a viral or non-viral delivery vector known in the art, such as electroporation or lipid nanoparticle. In other aspects, the DNA endonuclease may be delivered as one or more polypeptides alone, or delivered by pre-complexing with one or more guide RNAs, or one or more crRNAs as well as tracrRNAs. Some exemplary non-viral delivery vectors can be found in Peer and Lieberman, Gene Therapy, 18:1127-1133(2011).

The vector described herein may comprise the nucleic acid molecule of the present application (e.g., sequences encoding gRNAs, and/or gRNAs). The polynucleotide may be delivered by a non-viral delivery vector, including but not limited to nanoparticles, liposomes, ribonucleoproteins, positively charged peptides, small-molecule RNA conjugates, aptamer-RNA chimeras, and RNA fusion protein complexes. As described previously, the site-directed polypeptide and the genome-targeted nucleic acid each may be administrated to a cell or patient, respectively. In another aspect, the site-directed polypeptide may be pre-complexed with one or more guide RNAs or one or more crRNAs as well as tracrRNAs. The pre-complex material may then be administrated to the cell or patient. This pre-complex material is called as ribonucleoprotein particle (RNP). The RNA is capable of generating a specific interaction with the polynucleotide (e.g., RNA or DNA). Although this property is utilized in many biological processes, it also comes with the risk of promiscuous interactions in the nucleic acid-rich cellular environment. One way to address this problem is to form the ribonucleoprotein particle (RNP), in which RNA is pre-complexed with the nuclease. The RNP may protect the RNA from degradation. The nuclease in the RNP may be modified or unmodified. Likewise, the gRNA, crRNA, tracrRNA, or sgRNA may be modified or unmodified. There are many useful modifications known in the art, such as deletions, insertions, translocations, inactivations, and/or activations of the nucleotides. Such modifications may include introducing one or more mutations (including single or multiple base pair changes), increasing the number of hairpins, cross-linking, breaking specific stretches of nucleotides, and other modifications. The modifications may include the inclusion of at least one non-naturally occurring nucleotides, or modified nucleotides, or analogs thereof. The nucleotide may be modified at the ribose, phosphate, and/or base moieties.

The vector may also be a polynucleotide vector, such as plasmids, cosmid, or transposons. The suitable vectors for use have been widely described and are well known in the art. Those skilled in the art will appreciate that the vector comprising the nucleic acid molecule described herein may also comprise other sequences and elements useful and required for replication of the vector in prokaryotic and/or eukaryotic cells. For example, the vector described herein may comprise prokaryotic replicons, i.e., nucleotide sequences having the ability to direct the replication and maintenance of the host itself in a prokaryotic host cell (e.g., bacterial host cell). Such replicons are well known in the art. In certain instances, the vector may comprise shuttle elements that render the vector suitable for replication and integration in prokaryotes and eukaryotes. Moreover, the vector may also comprise a gene capable of expressing a detectable marker (e.g., drug resistance gene). The vector may also have a reporter gene, for example, a gene encoding a fluorescent protein or other detectable proteins.

In certain instances, the vector may include viral vectors, e.g., AAV, lentivirus, retrovirus, adenovirus, herpes virus, and hepatitis virus. The methods for producing the viral vector comprising the nucleic acid molecule (e.g., isolated nucleic acid molecule described herein) as part of the vector genome are well known in the art and may be performed by those skilled in the art without undue experimentation. In other instances, the vector may be a recombinant AAV virion that packages the nucleic acid molecule described herein. The methods for producing the recombinant AAV may include introducing the nucleic acid molecule described herein into a packaging cell line, producing the AAV infection as well as helper functions of the AAV cap and rep genes, and recovering the recombinant AAV from the supernatant of the packaging cell line. Various types of cells may be used as packaging cell lines. For example, the packaging cell lines that can be used include, but are not limited to, HEK 293 cells, HeLa cells, and Vero cells.

In some instances, the vector may be an adeno-associated virus (AAV) vector. In the present application, the term “adeno-associated virus vector” generally refers to the vectors derived from naturally occurring and available adeno-associated viruses as well as artificial AAVs. The AAV may include different serotypes, such as AAV1, AAV2, AAV3, AAV4, AAV5, AAV6, AAV7, AAV8, AAV9, AAV10, AAV11, AAV12, or AAV13, as well as any AAV variant or mixture. The AAV genome usually has inverted terminal repeats (ITRs) at both ends. The term “ITR” or “inverted terminal repeats” refers to a stretch of nucleic acid sequence present in the AAV and/or recombinant AAV, which can be form a T-shaped palindrome required for the completion of AAV lysis and latency lifecycle. The techniques for producing the AAV vector are standard techniques in the art, and include providing to cells the polynucleotide to be delivered, the rep and cap genes, and the AAV genome to be packaged for helper virus function. The production of AAV vector typically requires the presence of the following components within a single cell (herein referred to as packaging cell): rAAV genome, AAV rep and cap genes separate from (e.g., not in) the rAAV genome, as well as helper virus. The AAV rep and cap genes may be from any AAV serotype, and may also be from an AAV serotype different from the ITR of AAV genome, including but not limited to the AAV serotypes described herein. The AAV vector described herein may comprise the gRNA targeting the mutation site of the RHO gene. For example, the sequence encoding the gRNA may comprise a nucleotide sequence set forth in any of SEQ ID NOs. 1, 2, and 4.

In some instances, the sequence encoding the gRNA may be located in the same vector as the nucleic acid encoding the Cas9 protein. In other instances, the sequence encoding the gRNA may be located in a different vector than the nucleic acid encoding the Cas9 protein.

The AAV vector of the present application may be derived from a variety of species. For example, the AAV may be avian AAV, bovine AAV, or goat AAV. In certain embodiments, the vector is AAV8.

The method of the present application may include producing packaging cells, i.e., cell lines producing all necessary components for stably expressing the AAV. For example, an AAV genome lacking AAV rep and cap genes, AAV rep and cap genes isolated from the AAV genome, and a plasmid (or plasmids) with a selectable marker such as neomycin resistance gene are integrated into the genome of the cell. The AAV genome has been introduced into a bacterial plasmid by methods such as GC tailing (Samulski et al., 1982, Proc.Natl.Acad.S6.ETSA, 79:2077-2081). The packaging cell line may then be infected with a helper virus (e.g., adenovirus). In addition to the plasmid, the adenovirus or baculovirus may also be used to introduce the AAV genome and/or rep and cap genes into packaging cells.

In the present application, the term “subject” generally refers to any subject for whom the diagnosis, treatment, or therapy is desired. For example, in the present application, the subject in need thereof may have an RHO gene comprising a mutation site selected from the group consisting of c.C50T and c.C403T. In certain instances, the subject may include mammals. In certain instances, the subject may include humans. In certain instances, the subject may include East Asians.

In another aspect, the subject application provides a composition for treating retinitis pigmentosa in a subject, wherein the composition may comprise an active ingredient that removes a mutation site in an RHO gene, and a pharmaceutically acceptable carrier, wherein the mutation site is selected from the group consisting of c.C50T and c.C403T. The composition may comprise a physiologically tolerable carrier as well as a cellular composition, and optionally at least one bioactive agent dissolved or dispersed in the therapeutic composition as active ingredient. Generally, the carrier described herein may be administrated as suspension with a pharmaceutically acceptable carrier. Those skilled in the art will recognize that the pharmaceutically acceptable carrier that can be used may include buffers, compounds, cryopreservatives, preservatives, or other agents, which do not interfere with the delivery of the carrier. The composition may also comprise cell preparations, e.g., osmotic buffers which allow for the maintenance of cell membrane integrity, and optionally nutrient solutions which maintain cell viability or enhance the implantation upon administration. The formulations and suspensions are known to those skilled in the art, or can be adapted for use with the vectors and/or cells of the present application using routine experimentation.

In the present application, the term “administration” can introduce cells and/or vectors into a subject or certain desired site in the subject by a method or route. The cells and/or vectors can express the nucleic acid molecule described herein (e.g., the sequence encoding the gRNA, and/or the gRNA) at a desired site (e.g., a damage or repair site) to produce desired effects. The cells (or differentiated progeny thereof) and/or vectors may be administrated by any suitable route that can deliver the cells (or differentiated progeny thereof) and/or vectors to the desired site in the subject, and at least a portion of implanted cells (or cellular components) and/or carriers remain viable. After administration to a subject, the survival period of the cells may be as short as a few hours, e.g., twenty-four hours, several days, up to several years, or even corresponding to the lifespan of the patient. In certain instances, the administration comprises injection. For example, the vector may be administrated by a systemic route of administration, such as intraperitoneal or intravenous route. For example, the administration may include injection in the subretinal space.

The cleavage efficiency of the gRNA and vector described herein against the RHO allele mutation site may be about 50% or more, for example, about 55% or more, about 60% or more, about 65% or more, about 70% or more, about 75% or more, about 80% or more, about 85% or more, about 90% or more, about 95% or more, or about 98% or more, as detected in an in vitro enzymic cleavage reaction. For example, using a fluorescence detection kit, the gRNA and vector described herein can cleave the target nucleic acid with certain cleavage efficiency and specificity. In addition, the gRNA and vector described herein do not affect the survival of host cells, and have a certain safety.

Without intention to be limited by any theory, the following Examples are only intended to illustrate the fusion proteins, preparation methods, uses, etc. in the present application, and are not intended to limit the scope of the claimed invention.

### EXAMPLE

### Example 1. Information Collection on the Genetic Diagnosis of Patients

In this study, among more than 1,000 RP patients who visited in Peking University Third Hospital in recent years, a number of RP pedigrees with RHO gene mutations confirmed by gene diagnosis were collected. Two mutation hotspots in the RHO gene were found, as shown in Table 2 below.

TABLE 2
Mutation hotspots of RHO

| | | Nucleic acid | Amino acid | Whether the disease |
|---|---|---|---|---|
| Pedigree | Exon | change | change | has been reported |
| Mutation | E1 | c.50C > T | p.Thr17Met | Yes |
| hotspot 1 | | | | |
| Mutation | E2 | c.403C > T | p.Arg135Trp | Yes |
| hotspot 2 | | | | |

### Example 2. SgRNA Design and pX601-SgRNA Plasmid Construction

For the above 2 mutation sites, we designed a total of 5 sgRNAs for the SaCas9 system using Benchling website. The nucleotide sequences were shown in Table 3 below.

TABLE 3
SgRNA sequence

| Nucleic | | | Mutation | | SEQ | Sequence | SEQ |
|---|---|---|---|---|---|---|---|
| acid | Amino acid | SgRNA | position in | PAM | ID | encoding | ID |
| change | change | name | SgRNA | region | NO | gRNA | NO |
| c.50C>T | p.Thr17Met | RHO17- | 13^{rd} base at | GAGAA | 39 | CGTACCACACC | 1 |
| | | SgRNA1 | the 5′ end | | | CATCGCATTG | |
| | | RHO17- | 15^{th} base at | TGGAG | 40 | TGCGTACCACA | 2 |
| | | SgRNA2 | the 5′ end | | | CCCATCGCAT | |
| | | RHO17- | 16^{th} base at | TTGGA | 41 | CTGCGTACCAC | 3 |
| | | SgRNA3 | the 5′ end | | | ACCCATCGCA | |
| c.403C>T | p.Arg135Trp | RHO135- | 12^{th} base at | CAGGA | 42 | CACCACGTACC | 4 |
| | | SgRNA1 | the 5′ end | | | ACTCGATGGC | |
| | | RHO135- | 2^{nd} base at | AAGGA | 43 | CACTCGATGGC | 5 |
| | | SgRNA2 | the 5′ end | | | CAGGACCACC | |

The targeting vector used in the patent was: pX601-AAV-CMV::NLS-SaCas9-NLS-3xHA-bGHpA;U6::BsaI-sgRNA, which map was shown in FIG. 1 (for vector information, please refer to https://www.addgene.org/61591/).

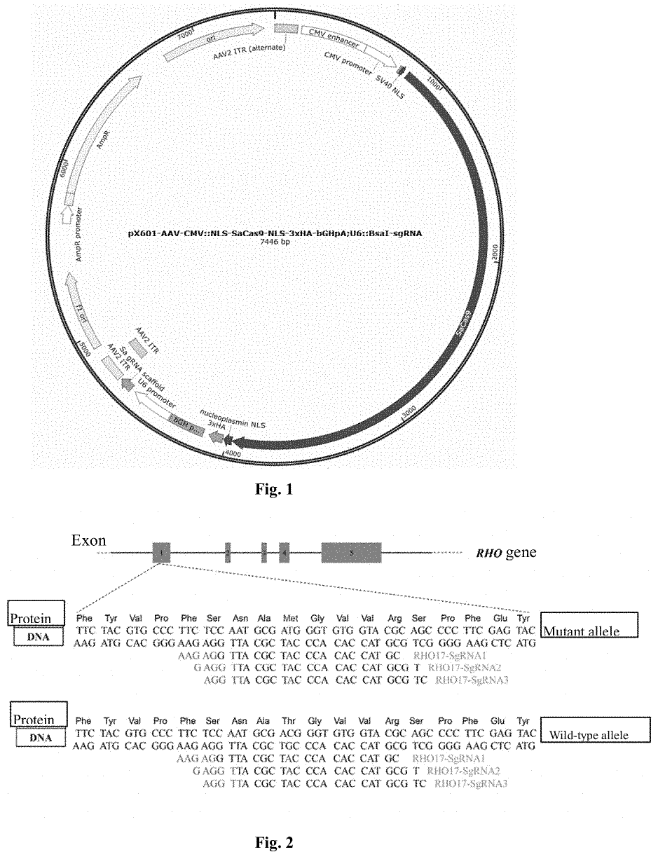

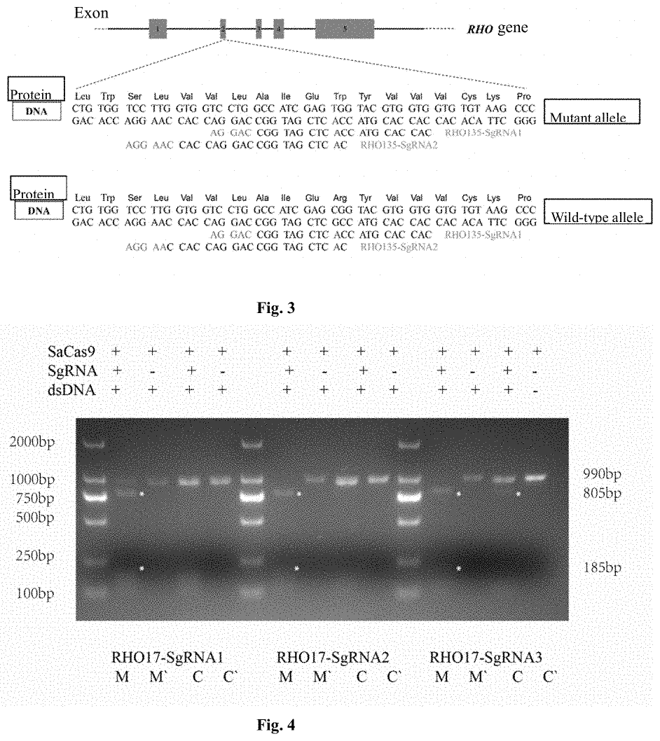

The schematic diagram of the sgRNA designed for RHO p.Thr17Met was shown in FIG. 2, and the schematic diagram for the sequence encoding the gRNA designed for RHO p.Arg135Trp was shown in FIG. 3.

The specific protocols for plasmid construction were as follows:

(1) SgRNA Annealing

T4 PNK and 10×T4 Ligation Buffer were thawed on ice for later use. The following reaction system was prepared:

TABLE 4
Reaction system

| | |
|---|---|
| DNA sequence mixture encoding the gRNA | 7 μl |
| 10× T4 Ligation Buffer | 1 μl |
| T4 PNK | 2 μl |
| Total | 10 μl |

The reaction system prepared as above was placed in a PCR instrument, and the following reaction program was run:

TABLE 5
Reaction program

| 37° C. | 30 min |
|---|---|
| 95° C. | 5 min |
| Ramp down to 25° C. at 5° C./min | |
| 25° C. | ∞ |

(2) Vector Digestion

BSaI digestion was used to release the binding site for the DNA sequence encoding the gRNA. The following digestion reaction system was prepared in a 1.5 ml PCR tube:

TABLE 6
Reaction system

| Plasmid (15 μg) | X | μl |
|---|---|---|
| 2× Cutsmart Buffer | 30 | μl |
| Enzyme e | 12 | μl |
| H₂O | 258-X | μl |
| Total | 300 | μl |

After digestion for 1-2 h/(K digestion overnight), the system was recovered and purified. The concentration was determined, and then it was diluted to 50 ng/μl.

(3) Ligation

The recovered vector from the previous step and the annealed DNA sequence encoding the gRNA were used to prepare the following ligation system (200 μl PCR tube):

TABLE 7
Ligation system

| | |
|---|---|
| Digestion vector | 1 μl |
| Annealed DNA sequence encoding the gRNA | 1 μl |
| 2× T4 Ligation Buffer | 5 μl |
| T4 Ligase | 1 μl |
| H₂O | 3 μl |
| Total | 11 μl |

The ligation reaction system from the previous step was placed at 37° C. for about 1-2 hours to complete the vector construction of the DNA sequence encoding the gRNA.

(4) Plasmid Transformation

1) The competent cells were placed on ice for thawing.

2) 1 μl of ligation system was added to 50 μl of competent cells, and kept on ice for half an hour, at 42° C. for 90 s, and then on ice for 2 min.

3) 500 μl of antibiotic-free medium was added and shaken for 1 h.

4) 100 μl was taken for plating.

5) On the next day, the colony was picked (500 μl of medium) and shaken for 3-4 hours, 200 μl of which was sent for sequencing.

(5) Plasmid extraction (in accordance with Omega Endo-Free Plasmid Maxiprep Kit)

1) The px601-SaCas9-RHO-SgRNA confirmed correct by sequencing was shaken overnight (50-200 mL), and incubated on a shaker at 37° C. for 12-16 h to amplify the plasmid, and extract on the next day (shaking for less than 16 h).

2) 50-200 mL of bacteria was centrifuged at 4,000×g for 10 min at room temperature, to collect the bacterial cells.

3) The medium was discarded. 10 mL of Solution I/RNaseA mixture was added to the pellet, and the cells were completely resuspended by pipetting or vortexing.

4) 10 mL of Solution II was added. The centrifuge tube was capped and gently inverted for 8-10 times to obtain a clear lysate. If necessary, the lysate was left to stand at room temperature for 2-3 minutes.

5) 5 mL of pre-chilled N3 Buffer was added. The centrifuge tube was capped and gently inverted for 10 times until a white flocculent precipitate was formed. The system may be left to stand and incubated at room temperature for 2 min.

6) A syringe filter was prepared. The plunger in the syringe was pulled out. Then the syringe was vertically placed on a suitable test-tube support, and a collection tube was placed at the outlet of the lower end of the syringe, with the opening of the syringe faced upward. The lysate was immediately poured into the syringe of the filter. The cell lysate was remained in the syringe for 5 min. At this time, white floccules would float on the surface of the lysate. The cell lysate may have flowed out of the filter syringe port. The cell lysate was collected in a new 50 mL tube. The plunger of the syringe was carefully and gently inserted into the syringe, and pushed slowly such that the lysate flowed into the collection tube.

7) One-tenth volume of ETR Solution (blue) was added to the filtered lysate that had flowed out. The tube was inverted for 10 times, and then left to stand in an ice bath for 10 minutes.

8) The above lysate was kept in water bath at 42° C. for 5 min. The lysate would be cloudy again. At this time, the lysate was centrifuged at 4,000×g for 5 minutes at 25° C. The ETR Solution would form a blue layer at the bottom of the tube.

9) The supernatant was transferred to another new 50 mL tube, and a half volume of absolute ethanol at room temperature was added. The tube was gently inverted for 6-7 times, and placed at room temperature for 1-2 min.

10) A HiBind® DNA Maxi binding column was cased in a 50 mL collection tube, and 20 mL of filtrate was added to the HiBind® DNA Maxi binding column, for centrifuging at 4,000×g for 3 min at room temperature. The filtrate was discarded.

11) The HiBind® DNA Maxi binding column was cased in the same collection tube to repeat step 10) until all the remaining filtrate was bound to the HiBind® DNA Maxi binding column, for centrifuging under the same conditions.

12) The HiBind® DNA Maxi binding column was cased in the same collection tube, and 10 mL of HBC Buffer was added to the HiBind® DNA Maxi binding column, for centrifuging at 4,000×g for 3 min at room temperature. The filtrate was discarded.

13) The HiBind® DNA Maxi binding column was cased in the same collection tube, and 15 mL of DNA Wash Buffer (diluted with absolute ethanol) was added to the HiBind® DNA Maxi binding column, for centrifuging at 4,000×g for 3 min at room temperature. The filtrate was discarded. Note: the concentrated DNA Wash Buffer must be diluted with ethanol according to the instructions before use. If the DNA Wash Buffer was refrigerated prior to use, it must be taken out and left at room temperature.

14) The HiBind® DNA Maxi binding column was cased in the same collection tube, and 10 mL of DNA Wash Buffer (diluted with absolute ethanol) was added to the HiBind® DNA Maxi binding column, for centrifuging at 4,000×g for 3 min at room temperature. The filtrate was discarded.

15) The matrix of the HiBind® DNA Maxi binding column (empty) was dried by centrifuging in an empty state at maximum speed (not exceeding 6,000×g) for 10 minutes.

16) (Optional) The HiBind® DNA Maxi binding column was further air-dried. One of the following processes was (optionally) chosen to further dry the HiBind® DNA Maxi binding column before the elution of DNA (if necessary):

a) The HiBind® DNA Maxi binding column was placed in a vacuum container for 15 min to dry the ethanol: the column was moved to the vacuum chamber at room temperature and all vacuum chamber devices were connected. The vacuum chamber was sealed and vacuumed for 15 min. The HiBind® DNA Maxi binding column was removed for the next operation. b) The column was dried in a vacuum oven or at 65° C. for 10-15 min. The HiBind® DNA Maxi binding column was removed for the next operation.

17) The HiBind® DNA Maxi binding column was placed in a clean 50 mL centrifuge tube, 1-3 mL of Endo-Free Elution Buffer was directly added onto the HiBind® DNA Maxi binding column matrix (the amount added depending on the expected final product concentration), and left to stand at room temperature for 5 min.

18) The DNA was eluted by centrifuging at 4,000×g for 5 min.

19) The column was discarded, and the DNA product was stored at −20° C.

The constructed pX601-SgRNA plasmid vector was named as follows:

TABLE 8
Plasmid vector

| Amino acid | Protein | | |
|---|---|---|---|
| change | change | SgRNA | Plasmid |
| c.50C > T | p.Thr17Met | RHO17-SgRNA1 | pX601-R17-sg1 |
| | | RHO17-SgRNA2 | pX601-R17-sg2 |
| | | RHO17-SgRNA3 | pX601-R17-sg3 |
| c.403C > T | p.Arg135Trp | RHO135-SgRNA1 | pX601-R135-sg1 |
| | | RHO135-SgRNA2 | pX601-R135-sg2 |

### Example 3. Determination of In Vitro Editing Efficiency of Each sgRNA for RHO by SaCas9-sgRNA Targeting Efficiency Detection Kit

The specific experimental protocols were as follows:

(1) In Vitro Transcription of sgRNA:

1) Primer design for in vitro transcription of sgRNA

TABLE 9
Primer sequences for in vitro
transcription of sgRNA

| | | SEQ |
|---|---|---|
| | | ID |
| Primer | Primer sequence | NO |
| RHO17 | TAATACGACTCACTATAGCGTACCACACCC | 6 |
| SgRNA1-T7 | ATCGCATTGGTTTTAGTACTCTGGAAACAG | |
| forward primer | | |
| RHO17 | TAATACGACTCACTATAGTGCGTACCACAC | 7 |
| SgRNA2-T7 | CCATCGCATGTTTTAGTACTCTGGAAACAG | |
| forward primer | | |
| RHO17 | TAATACGACTCACTATAGCTGCGTACCACA | 8 |
| SgRNA3-T7 | CCCATCGCAGTTTTAGTACTCTGGAAACAG | |
| forward primer | | |
| RHO135 | TAATACGACTCACTATAGCACCACGTACCA | 9 |
| SgRNA1-T7 | CTCGATGGCGTTTTAGTACTCTGGAAACAG | |
| forward primer | | |
| RHO135 | TAATACGACTCACTATAGCACTCGATGGCC | 10 |
| SgRNA2-T7 | AGGACCACCGTTTTAGTACTCTGGAAACAG | |
| forward primer | | |
| Reverse primer | ATCTCGCCAACAAGTTGACGAG | 11 |

2) Template Construction for in vitro transcription of sgRNA

The PCR reaction system was as follows:

TABLE 10
Reaction system

| Reagent | Volume (μL) |
|---|---|
| 2× KOD-FX Buffer | 25 |
| 2 mM dNTP mix | 10 |
| Forward prime | 1.5 |
| Reverse primer | 1.5 |
| px601 plasmid | 0.5 |
| KOD-FX DNA polymerase | 2 |
| Nuclease-Free H₂O | To 50 |

The PCR reaction program was as follows:

| | | | | |
|---|---|---|---|---|
| 94° C. | 2 | min | | |
| 98° C. | 10 | s | | |
| 58° C. | 30 | s | {close oversize brace} | 40 cycles |
| 68° C. | 30 | s | | |
| 68° C. | 5 | min | | |
| 16° C. | 10 | min | | |

3) 2.0% DNA gel was used for running the gel, and the gel recovery of the in vitro transcription template of the gRNA was performed using the OMEGA gel recovery kit in accordance with the following protocols:

a) An equal volume of membrane binding solution was added to the PCR reaction product (1 μL of membrane binding solution per 1 mg gel cutting and recovery), heated at 50-60° C. for 7 min until all the gels were completely dissolved, mixed by vortexing, and recovered by column;

b) The above liquid was dropped into the recovery column for centrifuging at 10,000×g for 1 min, and the filtrate was discarded;

c) 700 μL of Washing Buffer was added and centrifuged at >13,000×g for 1 min, and the filtrate was discarded;

d) Step c) was repeated;

e) The empty tube was centrifuged at >13,000×g for 10 min;

f) The centrifuge column was transferred to a new 1.5 mL Ep tube and marked. 20-30 μL of Elution Buffer or ddH₂O was added and kept at room temperature for 2 min;

g) After centrifuging at >13,000×g for 1 min, the adsorption column was discarded. The DNA was stored at 2-8° C., with measured and recorded concentration. It was required to be placed at −20° C. for long-term storage;

4) In vitro transcription of sgRNA (20 μL system):

TABLE 11
Transcription system

| Reagent | Volume (μL) |
|---|---|
| 10× Transcription Buffer | 2 |
| rNTP mixture | 2 |
| T7 RNA polymerase mixture | 2 |
| gRNA gel recovery template | 1 μg (≤14 μL) |
| DEPC H₂O | To 20 |

After mixing, the system was placed in a constant temperature incubator at 37° C. for reaction; after reaction completion, 2 μL of Dnase I was added to react at 37° C. for 30 min and then run the gel.

5) The gRNA was recovered in accordance with the instructions of the OMEGA gel recovery kit with the same protocols as above.

(2) Preparation of RHO sgRNA Cleavage Template

1) The genomic DNAs of induced pluripotent stem cells (iPSCs) (p.Thr17Met, p.Arg135Trp) derived from RHO-adRP patients and normal iPSCs without mutation sites were extracted, and used as the template to prepare the RHO sgRNA cleavage template dsDNA.

The genomic DNA extraction protocols were as follows:

a) The cells were collected by centrifugation at 400×g for 5 min, and the supernatant was discarded. 220 μl of PBS, 10 mL of RNase Solution, and 20 μL of PK working solution were added to the sample. The cells were resuspended, and left to stand at room temperature for more than 15 min.

b) 250 μl of Buffer GB was added to the cell resuspension, mixed well by vortexing, placed in water bath at 65° C. for 15-30 min, and purified by column;

c) 250 μl of absolute ethanol was added to the digestion solution, and mixed well by vortexing for 15-20 s;

d) The gDNA adsorption column was placed in a 2 ml collection tube. The mixture liquid obtained in the above step (including the precipitate) was transferred to the adsorption column, and centrifuged at 12,000×g for 1 min. If the column plugging occurred, the column was centrifuged at 14,000×g for 3-5 min. If the mixture liquid exceeded 750 μL, it was necessary to pass through the column in fractions.

e) The filtrate was discarded, and the adsorption column was placed in the collection tube. 500 μl of Washing Buffer A was added to the adsorption column, and centrifuged at 12,000×g for 1 min.

f) The filtrate was discarded, and the adsorption column was placed in the collection tube. 650 μl of Washing Buffer B was added to the adsorption column, and centrifuged at 12,000×g for 1 min.

g) Step 4 was repeated.

h) The filtrate was discarded, and the adsorption column was placed in the collection tube. The empty tube was centrifuged at 12,000×g for 2 min.

i) The adsorption column was placed in a new 1.5 ml centrifuge tube. 30-100 μl of Elution Buffer preheated to 70° C. was added to the center of the membrane of the adsorption column, left to stand at room temperature for 3 min, and then centrifuged at 12,000×g for 1 min.

Note: for DNA-rich tissues, 30-100 μl of Elution Buffer was further added to repeat the elution.

j) The adsorption column was discarded. The DNA was stored at 2-8° C., with measured and recorded concentration. It was required to be placed at −20° C. for long-term storage.

In this study, two groups of dsDNAs were required for each cleavage site. One group involved the dsDNA containing the mutation site, including RHO17-M-dsDNA and RHO135-M-dsDNA, with patient iPSC gDNA as template in the PCR process; the other group involved the dsDNA not containing the mutation site, including RHO17-C-dsDNA and RHO135-C-dsDNA, with normal human iPSC gDNA as template in the PCR process.

The primers used were shown in Table 12 below.

TABLE 12
Cleavage template primer sequences

| | | SEQ | PCR |
|---|---|---|---|
| | | ID | product |
| Primer | Primer sequence | NO | length |
| RHO17-forward | TTAGGAGGGGGAGGTCACTT | 12 | 990 bp |
| prime | TAT | | |
| RHO17-reverse | AAATCCACTTCCCACCCTGA | 13 | |
| prime | GC | | |
| RHO135-forward | GCCCAGAGCGCTAAGCAAAT | 14 | 835 bp |
| prime | A | | |
| RHO135-reverse | ATCGCCTAGAGGCTGAGTGG | 15 | |
| prime | | | |

2) The PCR reaction system was as follows:

TABLE 13
Reaction system

| Reagent | Volume (μL) |
|---|---|
| DNA | 0.4 |
| 5× PrimeSTAR Buffer | 10 |
| dNTP mixture | 4 |
| Forward prime | 3 |
| Reverse prime | 3 |
| PrimeSTAR HS DNA polymerase | 0.5 |
| DEPC treated water | 29.1 |

The PCR reaction program was as follows:

| | | | | |
|---|---|---|---|---|
| 95° C. | 2 | min | | |
| 95° C. | 10 | s | | |
| 56° C. | 30 | s | {close oversize brace} | 30 cycles |
| 72° C. | 30 | s | | |
| 72° C. | 5 | min | | |
| 16° C. | 10 | min | | |

3) 1.5% DNA gel was used for gel electrophoresis, and the PCR product was recovered using the OMEGA gel recovery kit in accordance with the same protocols as above:

(3) In Vitro Digestion Reaction for SaCas9-SgRNA

The reaction system was as follows:

TABLE 14
Reaction system

| Reagent | Volume (μL) |
|---|---|
| SaCas9 | 1 |
| 10× SaCas9 buffer | 2 |
| gRNA (in vitro transcription) | 50 ng |
| RHO sgRNA cleavage template | 50 ng |
| DEPC H₂O | To 20 |

The system was mixed well and reacted at 37° C. for 30 min 3 μL of DNA Loading Buffer was added, mixed, and boiled at 65° C. for 5 min A 2% agarose gel was run to analyze the digestion results.

The results of the in vitro efficiency determination of RHO17-SgRNA were shown in FIG. 4. Both RHO17-SgRNA1 and SgRNA2 could cleave the in vitro template of the experimental group, with more than 50% of cleavage efficiency for SgRNA1 and nearly 100% of cleavage efficiency for SgRNA2. The in vitro template of the control group was not cleaved, demonstrating that these two sgRNAs were excellent in both the cleavage efficiency and specificity, and could be applied in subsequent cell experiments and in vivo experiments. While RHO17-SgRNA3 cleaved the templates in both the experimental group and the control group, and thus could not be used due to the poor specificity. In FIG. 4, M: experimental group; C: control group (wild-type dsDNA).

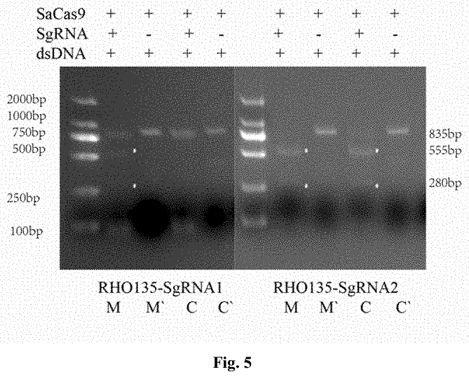

The results of the in vitro efficiency determination of RHO135-SgRNA were shown in FIG. 5. RHO135-SgRNA1 could cleave the in vitro template of the experimental group, with nearly 50% of cleavage efficiency. The in vitro template of the control group was not cleaved, demonstrating that this sgRNA was practicable in both the cleavage efficiency and specificity, and could be applied in subsequent cell experiments and in vivo experiments. While RHO135-SgRNA2 cleaved the templates in both the experimental group and the control group, and thus could not be used due to the poor specificity. In FIG. 5, M: experimental group; C: control group (wild-type dsDNA).

### Example 4. In Vitro Determination of gRNA Editing Efficiency Using a gRNA Activity Fluorescence Assay Kit

(1) Experimental Principle

The fluorescent reporter gene mKate in the fluorescent reporter plasmid of the kit was terminated prematurely by the stop codon. This truncated mKate was inactive. To detect the determination activity of gRNA, the target site recognized by Cas9/gRNA could be inserted after the stop codon. Then, under the action of Cas9 and gRNA, the double-stranded DNA at the target site was cleaved to form DSB, and an active fluorescent protein was formed in the cell through the homologous recombination effect. Whether the activity of the fluorescent protein had increased was detected by fluorescence microscope or flow cytometry, to judge the activity and knockout efficiency of gRNA.

(2) Design and Construction of Required Primers for Vector

The primer sequences were shown in Table 15.

TABLE 15
Primer sequences

| | | | SEQ ID |
|---|---|---|---|
| Primer | | Primer sequence | NO |
| RHO17-mKate- | forward | CTAGCGTACCACACCCATCGCATTGGAGAA | 16 |
| mut-sgRNA1 | reverse | GGCCTTCTCCAATGCGATGGGTGTGGTACG | 17 |
| RHO17-mKate- | forward | CTAGCGTACCACACCCGTCGCATTGGAGAA | 18 |
| wt-sgRNA1 | reverse | GGCCTTCTCCAATGCGACGGGTGTGGTACG | 19 |
| RHO17-mKate- | forward | CTAGTGCGTACCACACCCATCGCATTGGAG | 20 |
| mut-sgRNA2 | reverse | GGCCCTCCAATGCGATGGGTGTGGTACGCA | 21 |
| RHO17-mKate- | forward | CTAGTGCGTACCACACCCGTCGCATTGGAG | 22 |
| wt-sgRNA2 | reverse | GGCCCTCCAATGCGACGGGTGTGGTACGCA | 23 |
| RHO17-mKate- | forward | CTAGCTGCGTACCACACCCATCGCATTGGA | 24 |
| mut-sgRNA3 | reverse | GGCCTCCAATGCGATGGGTGTGGTACGCAG | 25 |
| RHO17-mKate- | forward | CTAGCTGCGTACCACACCCGTCGCATTGGA | 26 |
| wt-sgRNA3 | reverse | GGCCTCCAATGCGACGGGTGTGGTACGCAG | 27 |
| RHO135-mKate- | forward | CTAGCACCACGTACCACTCGATGGCCAGGA | 28 |
| mut-sgRNA1 | reverse | GGCCTCCTGGCCATCGAGTGGTACGTGGTG | 29 |
| RHO135-mKate- | forward | CTAGCACCACGTACCGCTCGATGGCCAGGA | 30 |
| wt-sgRNA1 | reverse | GGCCTCCTGGCCATCGAGCGGTACGTGGTG | 31 |
| RHO135-mKate- | forward | CTAGCACTCGATGGCCAGGACCACCAAGGA | 32 |
| mut-sgRNA2 | reverse | GGCCTCCTTGGTGGTCCTGGCCATCGAGTG | 33 |
| RHO135-mKate- | forward | CTAGCGCTCGATGGCCAGGACCACCAAGGA | 34 |
| wt-sgRNA2 | reverse | GGCCTCCTTGGTGGTCCTGGCCATCGAGCG | 35 |

(3) Experimental Protocols for Vector Construction

1) Primer annealing

The system was prepared using the synthesized primer sequences according to the following table. After annealing, DNA double-strands containing cohesive ends were generated.

TABLE 16
Reaction system

| | | |
|---|---|---|
| ddH₂O | 12 | μl |
| 10× Buffer | 2 | μl |
| 100 μM forward promer | 2 | μl |
| 100 μM forward promer | 2 | μl |
| Total | 20 | μl |

The reaction system prepared as above was placed in a PCR instrument, and the following reaction program was run:

TABLE 17
Reaction program

| 95° C. | 3 min |
|---|---|
| Ramp down to 25° C. at 5° C./min | |
| 25° C. | ∞ |

2) Vector ligation reaction

TABLE 18
Reaction system

| | |
|---|---|
| Annealing product | 1 μl |
| BG13701 vector | 1 μl |
| Solution I | 5 μl |
| ddH₂O | 3 μl |
| Total | 10 μl |

The reaction system prepared as above was placed in a PCR instrument, to run the following program: 16° C. 30 min-1 h.

3) Transformation: 5 μL of ligation product was added to 50 μL of DH5a competent cells that have just been thawed, and mixed well by flicking. The cells were placed in ice bath for 30 min, heat-shocked at 42° C. for 45 s, and then immediately left to stand on ice for 2 min. After adding 950 μL of LB liquid medium preheated at 37° C., the cells were cultured by shaking at 37° C. for 45 min. Then 100 μL of the cells were applied to an ampicillin-resistant plate.

4) Identification of positive clones: the designed forward sgRNA primer was paired with the sequencing primer TS-SP001, for colony PCR. The product size was 632 bp, and 2-3 positive colonies was picked and shaken. The plasmid DNA was extracted for sequencing. The complementary sequencing results should be reversed for the subsequent sequence alignment. The sequencing primer was TS-SP001: CTGATAGGCAGCCTGCACCTG (SEQ ID NO: 36). The sequencing sequence was as follows: vector sequence I (SEQ ID NO: 37)-sgRNA (SEQ ID NO: 1-5)-PAM (NNGRR(T), SEQ ID NO: 39)-vector sequence II (SEQ ID NO: 38).

5) The bacteria confirmed correct by sequencing was picked, and shaken overnight for plasmid extraction (in accordance with the same protocols as above).

(4) Plasmid Transfection and Analysis Results

1) The thawed 293T cells were placed in a 6-well plate.

2) The constructed plasmid containing the target sequence and the plasmid vector containing gRNA and Cas9 were co-transfected into the target cells. A negative control group was set.

3) The fluorescent signal was detected by fluorescence microscope: After transfection for 48 hours, the cells could be observed by flow cytometry. After comparing with the negative control group: if a stronger fluorescent signal was detected in the experimental group, it indicated that the activity of the gRNA was higher; and if the detected fluorescent signal was decreased in the experimental group or no fluorescent signal was detected in the experimental group, it indicated that the activity of the gRNA was weaker or inactive.

4) The activity of gRNA was detected by flow cytometry.

The constructed plasmid containing the target sequence and the plasmid vector containing gRNA and Cas9 were co-transfected into the target cells. A negative control group was set. The results of each group were triplicated for statistical analysis (two-tailed t-test, p<0.05).

The protocols of flow sorting were as follows:

a. The medium in the 6-well plate was aspirated and the cells were washed twice with DPBS;

b. 500 μl of 0.05% trypsin was added for incubating and digesting at 37° C. for 4 min;

c. 3-5 volumes of DMEM were added to neutralize the trypsin, and centrifuged at 800 r/min for 2 min;

d. The supernatant was aspirated, and PBS was added to resuspend the cells, and centrifuged at 800 r/min for 2 min; the process was repeated once;

e. The supernatant was aspirated, and 200 μl of PBS containing 2% FBS was added to resuspend the cells;

f. The obtained liquid in the above step was added to the filter tube, such that all the liquid passes through the filter screen;

g. The sample was placed in the instrument to run.

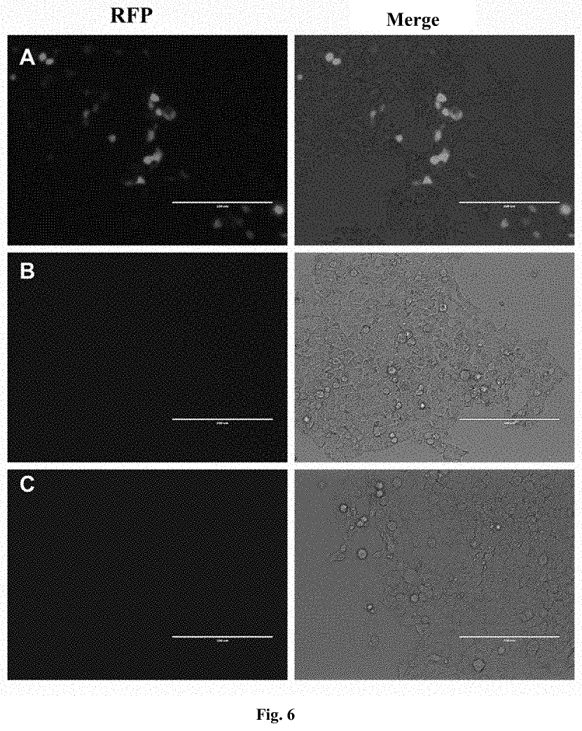

The results were shown in FIGS. 6 to 10.

FIG. 6 showed the in vitro determination of RHO17-SgRNA1 editing efficiency using the gRNA activity fluorescence assay kit. Panel A showed the experimental group, in which the co-transfected plasmids were RHO17-mkate-mut-sgRNA1 and pX601-R17-sg1, and RHO17-mkate-mut-sgRNA1 plasmid was cleaved at the target site by pX601-R17-sg1; Panel B showed RHO17-mkate-mut-sgRNA1 and pX601 empty plasmids in the experimental control group, in which there was no cleavage; Panel C showed the control group, in which the co-transfected plasmids were RHO17-mkate-wt-sgRNA1 and pX601-R17-sg1, and RHO17-mkate-wt-sgRNA1 plasmid was not cleaved at the target site by pX601-R17-sg1. The above results proved that identical to the results of the in vitro cleavage experiment as above, this sgRNA was practicable in both the cleavage efficiency and specificity, and could be applied in subsequent cell experiments and in vivo experiments.

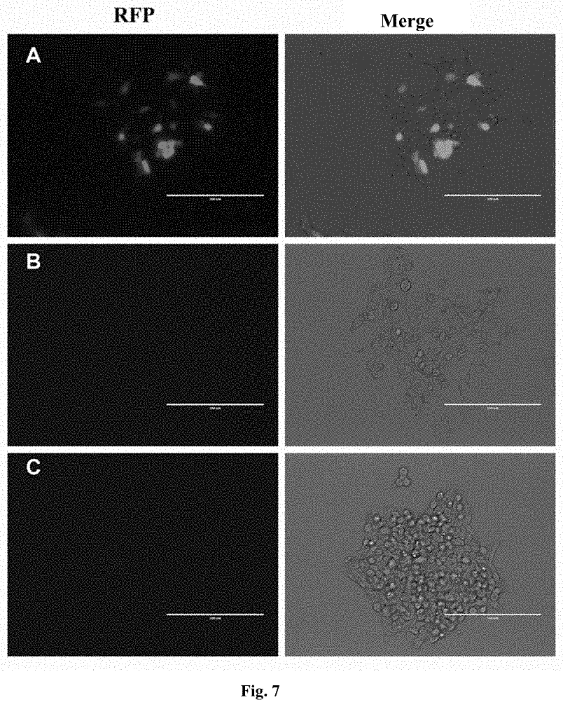

FIG. 7 showed the in vitro determination of RHO17-SgRNA2 editing efficiency using the gRNA activity fluorescence assay kit. Panel A showed the experimental group, in which the co-transfected plasmids were RHO17-mkate-mut-sgRNA2 and pX601-R17-sg2, and RHO17-mkate-mut-sgRNA2 plasmid was cleaved at the target site by pX601-R17-sg2; Panel B showed RHO17-mkate-mut-sgRNA2 and pX601 empty plasmids in the experimental control group, in which there was no cleavage; Panel C showed the control group, in which the co-transfected plasmids were RHO17-mkate-wt-sgRNA2 and pX601-R17-sg2, and RHO17-mkate-wt-sgRNA2 plasmid was not cleaved at the target site by pX601-R17-sg2. The above results proved that identical to the results of the in vitro cleavage experiment as above, this sgRNA was practicable in both the cleavage efficiency and specificity, and could be applied in subsequent cell experiments and in vivo experiments.

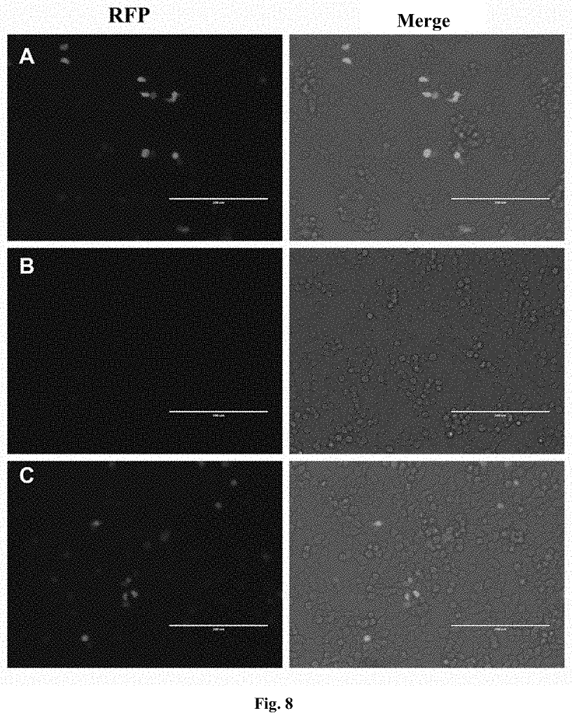

FIG. 8 showed the in vitro determination of RHO17-SgRNA3 editing efficiency using the gRNA activity fluorescence assay kit. Panel A showed the experimental group, in which the co-transfected plasmids were RHO17-mkate-mut-sgRNA3 and pX601-R17-sg3, and RHO17-mkate-mut-sgRNA3 plasmid was cleaved at the target site by pX601-R17-sg3; Panel B showed RHO17-mkate-mut-sgRNA3 and pX601 empty plasmids in the experimental control group, in which there was no cleavage; Panel C showed the control group, in which the co-transfected plasmids were RHO17-mkate-wt-sgRNA3 and pX601-R17-sg3, and RHO17-mkate-wt-sgRNA3 plasmid was cleaved at the target site by pX601-R17-sg3. The above results proved that identical to the results of the in vitro cleavage experiment as above, this sgRNA exhibited the worse cleavage specificity, and could not be applied in subsequent cell experiments and in vivo experiments.

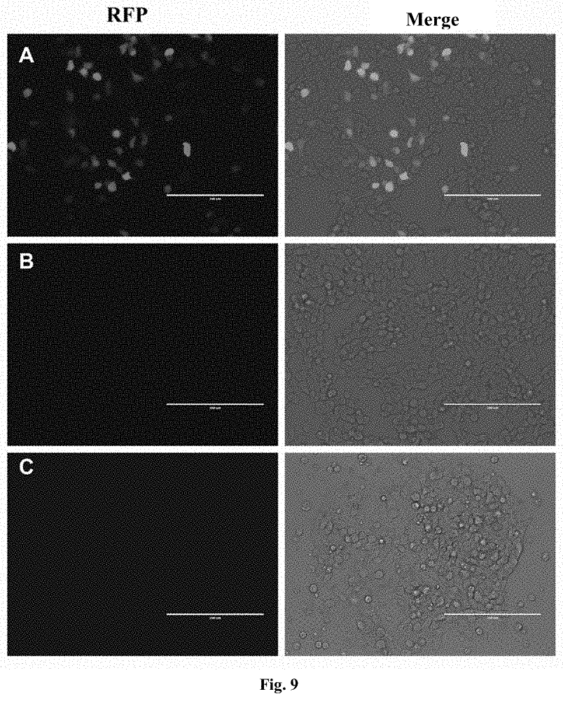

FIG. 9 showed the in vitro determination of RHO135-SgRNA1 editing efficiency using the gRNA activity fluorescence assay kit. Panel A showed the experimental group, in which the co-transfected plasmids were RHO135-mkate-mut-sgRNA1 and pX601-R135-sg1, and RHO135-mkate-mut-sgRNA1 plasmid was cleaved at the target site by pX601-R135-sg1; Panel B showed RHO135-mkate-mut-sgRNA1 and pX601 empty plasmids in the experimental control group, in which there was no cleavage; Panel C showed the control group, in which the co-transfected plasmids were RHO135-mkate-wt-sgRNA1 and pX601-R135-sg1, and RHO135-mkate-wt-sgRNA1 plasmid was not cleaved at the target site by pX601-R135-sg1. The above results proved that identical to the results of the in vitro cleavage experiment as above, this sgRNA was practicable in both the cleavage efficiency and specificity, and could be applied in subsequent cell experiments and in vivo experiments.

FIG. 10 showed the in vitro determination of RHO135-SgRNA2 editing efficiency using the gRNA activity fluorescence assay kit. Panel A showed the experimental group, in which the co-transfected plasmids were RHO135-mkate-mut-sgRNA2 and pX601-R135-sg2, and RHO135-mkate-mut-sgRNA2 plasmid was cleaved at the target site by pX601-R135-sg2; Panel B showed RHO135-mkate-mut-sgRNA2 and pX601 empty plasmids in the experimental control group, in which there was no cleavage; Panel C showed the control group, in which the co-transfected plasmids were RHO135-mkate-wt-sgRNA2 and pX601-R135-sg2, and RHO135-mkate-wt-sgRNA2 plasmid was cleaved at the target site by pX601-R135-sg2. The above results proved that identical to the results of the in vitro cleavage experiment as above, this sgRNA exhibited the worse cleavage specificity, and could not be applied in subsequent cell experiments and in vivo experiments.

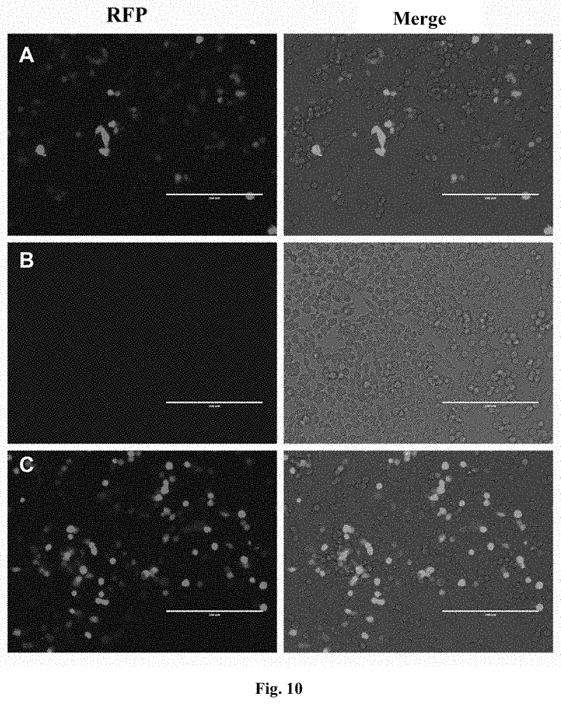

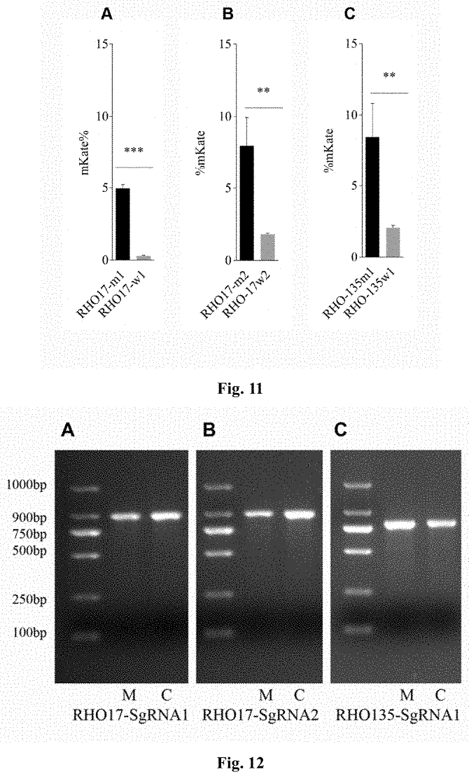

FIG. 11 showed the statistics of flow sorting results of the in vitro determination of sgRNA editing efficiency using the gRNA activity fluorescence assay kit. As can be seen from Panel A, the experimental group (RHO17-m1) and the control group (RHO17-W1) were statistically different in the cleavage efficiency; and as can be seen from Panel B, the experimental group (RHO17-m2) and the control group (RHO17-W2) were statistically different in the cleavage efficiency; and as can be seen from Panel C, the experimental group (RHO135-m1) and the control group (RHO135-W1) were statistically different in the cleavage efficiency; these could be used in the subsequent cell and in vivo experiments. Three replicate groups were set up in each of the experimental group and the control group for flow sorting.

### Example 5. In Vitro Validation of gRNA Safety and Specificity Using 293T Cells

(1) 293T Cell Culture

293T cells were cultured at 37° C. with 5% CO₂, using high-glucose DMEM supplemented with 10% fetal bovine serum and 100 U/ml penicillin/streptomycin as the medium.

1) Thawing of frozen cells

a) The temperature of the thermostatic water bath was adjusted to 37° C. The frozen cells were taken out from the liquid nitrogen. By clamping the lid with tweezers, the container was shaken quickly in the water.

b) The liquid was transferred to a 15 ml centrifuge tube, 10 ml of culture medium was slowly added, and shaken gently to homogenize the liquid. After tightening the lid, the tube was centrifuged at 1,000 rpm/min for 3 min.

c) Removing the supernatant and adding an appropriate amount of medium. The cell at the bottom was pipetted gently, and then the cells were transferred to a culture flask for culturing in an incubator.

2) Cell passage

a) The morphology and density of the cells were observed under an inverted microscope. When the cells in the culture flask reached 80%-90% confluence, the cells were passaged.

b) The old medium in the cell culture flask was washed out, and the flask was washed with PBS for 3 times. 500 μl of EDTA-containing trypsin was added to the culture flask, and incubated for about 1 min in the incubator. When the intercellular space became larger and the cells became round, 1 ml of medium was immediately added to the culture flask to stop the digestion. The cells were pipetted gently, and after all the cells floated from the bottom of the flask, the liquid in the culture flask was transferred to a centrifuge tube and centrifuged at 1,000 rpm/min for 2 min.

c) The supernatant was discarded, and 2 ml of medium was added to the centrifuge tube to resuspend the cells. The cell suspension was dispensed into 4 new culture flasks, and 4 ml of medium was added to each flask. The culture flask was shaken gently such that the cells were mixed evenly and plated onto the culture flask, and then placed into a cell incubator for culturing.

d) On 1-2 days before transfection, 293T cells were seeded into a 6-well plate, and subjected to plasmid transfection when the cells reached 80-90% confluence.

(2) 293T Cell Transfection

1) The pX601-SaCas9 plasmids constructed in Example 2 were numbered, as shown in the following table:

TABLE 19
Plasmid vector

| Amino acid change | Protein change | Plasmid name |
|---|---|---|
| c.50C > T | p.Thr17Met | pX601-R17-sg1 |
| | | pX601-R17-sg2 |
| | | pX601-R17-sg3 |
| c.403C > T | p.Arg135Trp | pX601-R135-sg1 |
| | | pX601-R135-sg2 |

2) The process and protocol for plasmid transfection were as follows:

1.5 mL EP tubes were numbered in the above order. 250 μL of DMEM medium (serum-free) was added to each tube, and 1.5 μg of pX601-RHO-SgRNA plasmid and 1 μg of pLenti-GFP plasmid were successively added according to the above table. After mixing well by vortexing, 7.5 μL of PEI transfection reagent was added to each tube, mixed well by flicking with fingers (instead of vortexing), and left to stand at room temperature for 20 min before transfection.

For the 6-well plate with cultured 293T cells, the cell confluence was observed under the microscope. The culture medium was changed by discarding the waste medium and adding 1.75 mL of new complete medium. Then the cells in each well were numbered as above. The prepared transfection system was added to each well according to numbering for culturing overnight.

On the next day, the GFP expression was observed under a fluorescence microscope to assess the transfection efficiency, and the transfected plasmid was continued to be cultured if the transfection efficiency was good.

Two days after transfection, an appropriate amount of puromycin was added to each well (including the negative control group) (note: the antibiotic concentration could be gradually increased from 0.1 μg/mL to 0.5 μg/mL) to screen for positively transfected cells. Then the survival state of the cells was observed every day. The culture medium was changed every 2 days, and a corresponding amount of puromycin was added when changing the medium.

At the time that the cells in the negative control well were completely dead while the experimental group and the control group had viable cells (indicating successful transfection), the antibiotic screening was terminated and the culture medium was changed to the normal medium. After the cells in the 6-well plate reached 80-90% confluence, the cells were passaged to a 6 cm culture dish. After the cells reached 80-90% confluence, the cells were harvested for extracting the genomic DNA. The whole process took about 7-10 days.

3) Extraction of Genomic DNA from 293T Cells

The protocols were the same as above.

(3) Validation of gRNA Editing Efficiency

1) T7E1 digestion experiment

The PCR program was run based on the above system. After gel electrophoresis, the PCR product was recovered in accordance with the same protocols as above.

The product obtained by PCR recovery or gel cutting recovery as above was subjected to the T7E1 digestion reaction.

a) Annealing system for T7E1 digestion (19.5 μL):

TABLE 20
Reaction system

| Reagent | Volume (μL) |
|---|---|
| NEB Buffer 2 | 2 |
| PCR or gel cutting recovery product | X (500 ng or 1,000 ng) |
| Deionized H₂O | To 19.5 |

b) Annealing program for T7E1 digestion:

95° C. 2 min

95° C. to 85° C. Temperature −2° C./s

85° C. to 25° C. Temperature −0.1° C./s

16° C. ∞.

b) Reaction system for T7E1 digestion

TABLE 21
Reaction system

| Reagent | Volume (μL) |
|---|---|
| Annealing product | 9.75 or 9.5 |
| T7E1 enzyme | 0.25 or 0.5 |

37° C. 20 min

d) Gel electrophoresis of digestion product

Gel formulation: 2.5% gel, with double dye added.

Gel electrophoresis program: 140 V, 20 min to 30 min.

e) The gel electrophoresis results were checked. The results were shown in FIG. 12. As shown in Panel A of FIG. 12, after transfecting 293T cells with pX601-R17-Sg1, the target genome (wild-type sequence) was not cleaved by this plasmid, indicating that RHO17-SgRNA1 had good specificity; as shown in Panel B of FIG. 12, after transfecting 293T cells with pX601-R17-Sg2, the target genome (wild-type sequence) was not cleaved by this plasmid, indicating that RHO17-SgRNA2 had good specificity; as shown in Panel C of FIG. 12, after transfecting 293T cells with pX601-R135-Sg1, the target genome (wild-type sequence) was not cleaved by this plasmid, indicating that RHO135-SgRNA1 had good specificity. In FIG. 12, M: pX601-R17-Sg1, pX601-R17-Sg2, or pX601-R135-Sg1; C: pX601.

### Example 6. Validation of gRNA Editing Efficiency Based on Patient-Derived iPSCs

(1) Extraction and Culture of Renal Epithelial Cells from Patients

In this application, patient A carried RHO c.50C>T mutation, patient B carried RHO c.403C>T, and normal person C neither suffered to the disease nor carried any gene mutation sites.

Using the renal epithelial cell isolation and culture kit provided by Beijing Cellapy, the experimental protocols were as follows:

1) The UrinEasy isolation complete medium, supplements, Gelatin, and washing solution were brought to the cell room. The supplements were thawed in the refrigerator and others were placed in room temperature.

2) The 12-well plate, 50 ml centrifuge tube, 15 ml centrifuge tube, electric pipettor, pipette, sucker, 5 ml pipettor and tips, and 1 ml pipettor and tips were irradiated under UV lamp; and a water bath kettle at 37° C. was turned on.

3) With gloves and disinfection, the urine (preferably midstream urine) was collected, and sealed with PARAFILM.

4) 750 μL/well of Gelatin was added to coat the bottom of the dish (3 wells) for not less than half an hour, and kept at 37° C.

5) The outer surface of the urine bottle was disinfected with 75% alcohol, and the urine was dispensed into a 50 mL conical-bottom centrifuge tube. The tube was sealed and centrifuged at 400×g for 10 min.

6) The UrinEasy isolation complete medium+supplements were removed and formulated (5 mL of basic medium per 0.5 mL of supplements).

7) Along the upper surface, the supernatant was slowly aspirated by pipette to 1 ml with the minimum speed.

8) The liquid was resuspended in a 15 ml centrifuge tube, 10 ml of washing solution was added and mixed well, then centrifuged at 200×g for 10 min.

9) Gelatin was aspirated from the 12-well plate, and the plate was washed once with the washing solution (500 μL). 750 μL of UrinEasy isolation complete medium was added and kept at 37° C.

10) The 15 ml centrifuge tube was taken out, and 0.2 ml of cell pellet remained.

11) The cell pellet was resuspended with UrinEasy isolation complete medium: one well for male and two wells for females, denoted as DO.

12) Observation:

D1: whether there had contamination was observed;

D2: the isolation medium was supplemented—female: 500 μl/well; male: 250 μl/well;

D4: If the cells were not adherence: half volume of the medium was changed every two days by slowly adding 1 mL of isolation complete medium along the wall.

13) Until the adherence occurred: after the cells appeared to adhere (3˜7 days or 9˜10 days), UrinEasy expansion complete medium was added.

The cells were cultured for two days, and the full volume (500 μL) of medium was changed. At approximately 9˜12 D (not exceeding 14 D) after adherence, the cells at 80-90% confluence were respectively passaged to 6-well plate, 6 cm dish, and 10 cm dish, and then frozen for later use.

(2) Induction of iPSCs

The patient-derived (p.Arg135Trp and p.Thr17Met) renal epithelial cells were induced into iPSCs in accordance with the following protocols:

1) The somatic cells were digested and passaged when the cells reached 70-90% confluence. The cells were seeded in a 96-well plate with a controlled density of 5,000-15,000 cells/well. 3 density gradients might be set according to the cell conditions, with 3 duplicate wells for each gradient. The day of cell seeding was recorded as day −1.

2) Day 0: The confluence and state of cells were observed under the microscope, and duplicate wells with different gradients were selected for digestion and counting. The wells with 10,000-20,000 cells were chosen for reprogramming. The Reprogramming Medium A was prepared according to the table below:

TABLE 22
Culture medium formulation

| Reprogramming Medium A | Volume | |
|---|---|---|
| Somatic Cell Culture Medium | 10 | mL |
| Reprogramming Supplement I | 10 | μL |

3) The Reprogramming Supplement II was first centrifuged, and then 97 μL of Reprogramming Medium A was added to the tube of Reprogramming Supplement II, and mixed well to prepare the Reprogramming Medium B. 100 μL of Reprogramming Medium B was added to a chosen eligible 96-well, and the plate was placed back into the incubator.

4) Days 1-2: The cells were observed under the microscope and photographed to record the changes in cell morphology. If the cell morphology was obviously changed, the Reprogramming Medium B was removed and replaced with the Reprogramming Medium A for further culturing. If the morphological changes were not obvious, the medium might not be changed.

5) Day 3: If the cell morphology had subjected to obvious deformation in the first two days, and the cell growth rate was relatively fast, the cells were digested by trypsin and passaged. Depending upon the cell state and amount, the cells were transferred to 2-6 wells of a 6-well plate, and the Reprogramming Medium C was added to form the single-cell adherence as much as possible. The reprogramming medium C was prepared according to the table below:

TABLE 23
Culture medium formulation

| Reprogramming Medium C | Volume |
|---|---|
| Reprogramming Medium A | 9.8 mL (remained as above) |
| Reprogramming Supplement III | 5 μL |

6) Day 4: The adherence of the cells was observed. If most of the cells adhered well, the medium was replaced with fresh somatic cell culture medium for further culturing.

7) Day 5: The cells were observed under the microscope. If a small clone cluster (clone pellet with more than 4 cells) was formed, the somatic cell culture medium was replaced with Reproeasy Human Somatic Cell Reprogramming Medium. If no small clone cluster was formed, the cells were further observed for one or two days before replacing the medium with the Reproeasy Human Somatic Cell Reprogramming Medium.

8) Days 6-8: The cells were observed under the microscope. If the small clone cluster became larger and there were more than 10 cells in a clone pellet, Reproeasy Human Somatic Cell Reprogramming Medium was directly replaced with PSCeasy Human Pluripotent Stem Cell Medium (or PGM1 Human Pluripotent Stem Cell Medium). If a relatively large number of dead cells were observed before changing the medium, the cells might be washed with PBS equilibrated at room temperature before changing the medium.

9) Days 9-20: The cells were observed under the microscope and photographed to record the changes in cell morphology. The fresh PSCeasy Human Pluripotent Stem Cell Medium equilibrated at room temperature was replaced daily.

10) Day 21: The cells were observed under the microscope. If a single cell clone could fill the entire 10× field of view, a 1 mL syringe needle (or other equipment such as glass needles) was used to cut and pick the clone into a 48-cell plate coated with Matrigel in advance (if the clone was in good condition with massive cells and fast growth, it could be directly picked into a 24-well plate).

11) The clone was picked out and seeded with PSCeasy Human Pluripotent Stem Cell Resuscitation Medium. After the cells adhered to the wall, the medium was replaced with PSCeasy Human Pluripotent Stem Cell Medium for further culturing to the desired passage.

(3) Electrotransfection of iPSCs

1) The pX601 plasmid was engineered to carry the resistance against puromycin for screening after electrotransformation. The engineered plasmid was named as pX601-R17-puro-sg 1, pX601-R17-puro-sg2, and pX601-R135-puro-sg1; and the blank control plasmid was pX601-GFP-puro plasmid.

2) The protocols for the electrotransfection of iPSCs were as follows:

a) The frozen iPSCs were thawed into a 6-well plate, and electrotransfected when the cells reached 60-80% confluence.

b) The medium was discarded, the cells were washed with 1 mL of DPBS, and the waste liquid was discarded. The process was repeated twice.

c) 1 mL of 0.05% EDTA digestion solution was added to the iPSCs, and left to digest at 37° C. for 3 min;

d) The 6-well plate was observed under the light microscope to confirm the cell digestion. The complete medium was added to neutralize the digestion solution, the wall of the dish was tapped gently, and the cells were gently pipetted by the pipette tip. The cell suspension was collected into a 15 mL centrifuge tube for counting, and centrifuged at room temperature at 200×g for 5 min.

e) The supernatant was discarded as much as possible. The cells were resuspended with 100 μL of prepared electrotransfection solution (82 μL basic electrotransfection solution+18 μL electrotransfection additive+5 μg plasmid). The number of all electrotransfection cells is 1×10⁶. The system was mixed well by pipetting gently, and aspirated by sucker to the bottom of the electrode cup to ensure that the bottom of the cup was completely covered and no air bubbles were generated. The electrode cup was placed in the Lonza electrotransfection instrument for electrotransfection with program CA-137.

f) After electrotransfection, the iPSCs were re-seeded into a 6-well plate for culturing.

g) 24 hours after transfection, the survival state of the cells was observed, and the cells were screened with puromycin on the second day after transfection.

h) The subsequent cell culture and screening were performed in accordance with Example 5.

(4) Validation of gRNA Editing Efficiency

1) Extraction of gDNA from iPSCs

The protocols were the same as above.

2) PCR reaction

The primers used were shown in Table 24 below in accordance with the same protocols as above.

TABLE 24
Primer sequences

| | | SEQ | PCR |
|---|---|---|---|
| | | ID | product |
| Primer | Primer sequence | NO | length |
| RHO17-forward | TTATGAACACCCCCAATCTCCC | 49 | 680 bp |
| primer | | | |
| RHO17-reverse | GAGGGCTTTGGATAACATTGAC | 50 | |
| primer | | | |
| RHO135-forward | GCCCAGAGCGCTAAGCAAATA | 14 | 835 bp |
| primer | | | |
| RHO135-reverse | ATCGCCTAGAGGCTGAGTGG | 15 | |
| primer | | | |

3) T7E1 Digestion Experiment

The protocols were the same as above.

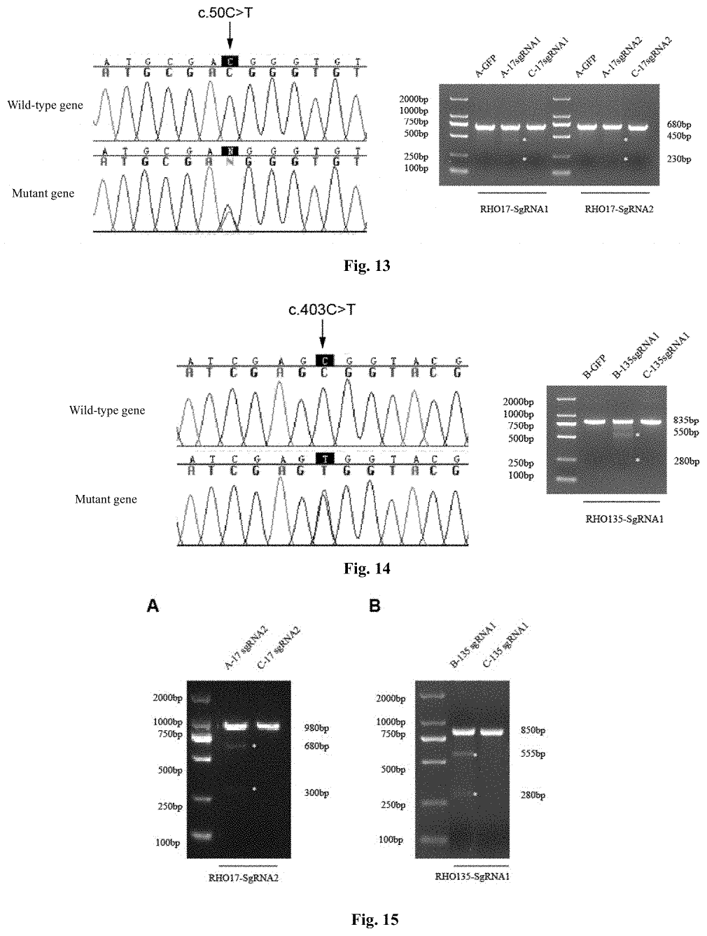

The experimental results of patient A were shown in FIG. 13. The Sanger sequencing results in the left panel showed that patient A carried the heterozygous mutation of RHO c.50C>T, while the normal person did not; the right panel showed the gene editing effects of RHO17-SgRNA1 and RHO17-SgRNA2 in the iPSCs of patient A and normal person C, wherein A-GFP indicated that the iPSCs of patient A were only transfected with pX601-GFP-puro blank control plasmid; A-17sgRNA1 or A-17sgRNA2 indicated that the iPSCs of patient A were transfected with pX601-R17-puro-sg1 or pX601-R17-puro-sg2 plasmid; C-17sgRNA1 or C-17sgRNA2 indicated that the iPSCs of normal person were transfected with pX601-R17-puro-sg1 or pX601-R17-puro-sg2 plasmid. The T7E1 results showed that RHO17-SgRNA1 and RHO17-SgRNA2 only had the editing effect on the RHO gene mutation site in the iPSCs of patient A, but had no editing effect at the corresponding gene site in the normal person. The results were consistent with those of the in vitro experiments as above.

The experimental results of patient B were shown in FIG. 14. The Sanger sequencing results in the left panel showed that patient B carried the heterozygous mutation of RHO c.403C>T, while the normal person did not; the right panel showed the gene editing effect of RHO135-SgRNA1 in the iPSCs of patient B and normal person C, wherein B-GFP indicated that the iPSCs of patient B were only transfected with pX601-GFP-puro blank control plasmid; B-135sgRNA1 indicated that the iPSCs of patient B were transfected with pX601-R135-puro-sg1 plasmid; C-135sgRNA1 indicated that the iPSCs of normal person were transfected with pX601-R135-puro-sg1 plasmid. The T7E1 results showed that RHO135-SgRNA1 only had the editing effect on the RHO gene mutation site in the iPSCs of patient B, but had no editing effect at the corresponding gene site in the normal person. The results were consistent with those of the in vitro experiments as above.

### Example 7. In Vitro Validation of gRNA Gene Editing Efficiency Using 3D Retinal Tissue

(1) The iPSCs Derived from Patient a, Patient B, and Normal Person C were Induced into 3D Retina Tissues.

Table 25 below shows the specific protocols:

TABLE 25
Experimental protocols

| | | | |
|---|---|---|---|
| Stage 1 | D1 | The iPSCs were seeded in a T25 flask at a density of 30,000/cm² | E8 |
| | | and cultured until the cell density reached about 70%. | medium |
| Stage 2 | D1 | The cells were digested with Dispase (2 mg/ml), and 3:1 | E8 + |
| | | E8:NIM + rock inhibitor (Y27632) medium was added to suspend | NIM |
| | | the cultured cells in a 10 cm dish. (EB was formed on the next | |
| | | day and existed alone) | |
| | D2 | The medium was changed (E8:NIM = 1:1). | |
| | D3 | The medium was changed (E8:NIM = 1:3). | |
| | D4-5 | The medium was changed (Complete NIM). | NIM |
| | D6 | The medium was changed (NIM + 50 ng/ml BMP4). | |
| | D7 | EBs with good morphology (single-chamber, clear) were chosen, | |
| | | and 1 ml (about 200 EBs) medium was dropwise added to the | |
| | | adherent 6-well plate, and then (1 ml NIM + 250 μl FBS)/well was | |
| | | added. | |
| | D8 | The medium was changed ((4 ml NIM + 25 ng/ml BMP4)/well). | |
| | D9-15 | The medium was changed every 1.5 day (NIM). | |
| Stage 3 | D16 | The aggregated cell pellets were transferred into a 15 ml | RDM |
| | | centrifuge tube with a 10 μl pipette tip, such that they sunk to the | |
| | | bottom naturally. The supernatant was removed, and 5 ml RDM | |
| | | was added to resuspend. The suspension was seeded in a 6 cm | |
| | | dish, and the medium was changed every 2-3 days. | |
| | D18-0 | By observation under 4 × x microscope, the retinal neurospheres | |
| | | (pellets) with bright outer rings on the surface were collected | |
| | | gently and slowly, and transferred to a 15 ml centrifuge tube | |
| | | containing 5 ml RDM. The collected retinal neurospheres | |
| | | (pellets) were transferred into a 6 cm dish. | |
| | D20-30 | The medium was changed every 2-3 days. Continue to select | |
| | | The retinal organoids were continually chosen based on the | |
| | | presence or absence of bright outer ring in the appearance. | |
| | | During this period, some organoids might be gradually | |
| | | transformed towards non-retinal neurospheres? After 30 days, | |
| | | the remaining retinal neurospheres could maintain their | |
| | | morphology ever since. (When the organoids were aggregated | |
| | | together, they should be separated with sterile nippers as soon as | |
| | | possible) | |

(2) Construction and Coating of AAV8 Virus

RHO17-SgRNA1 and RHO17-SgRNA2 edited the same site. In view of the results of in vitro experiments and stem cell experiments, the latter had a stronger cleavage effect than the former. RHO17-SgRNA2 was used in both 3D retina experiment and mouse experiment use RHO17-SgRNA2.

The pX601 plasmid as above was engineered to carry the GFP fluorescent protein for subsequent screening of GFP+ cells. The engineered plasmids were named as pX601-R17-GFP-sg2 and pX601-R135-GFP-sg1, and the blank control plasmid was pX601-GFP plasmid.

1) Plasmid amplification. The constructed AAV vector, packaging plasmid, and helper plasmid should be subjected to the endo-free maxiprep extraction. The Qiagen maxiprep extraction kit was used for the maxiprep plasmid extraction in accordance with the same protocols as above.

2) AAV8-293T cell transfection. The cell density was observed on the day of transfection, and at 80-90% confluence, the vector plasmid, packaging plasmid, and helper plasmid were used for transfection.

3) AAV8 virus collection. The virus particles were existed in both packaging cells and culture supernatant. Both the cells and the culture supernatant could be collected for the best yield.

4) AAV was purified and stored at −80° C. for long-term storage.

(3) 3D Retinal Organoid In Vitro Validation of gRNA Editing Efficiency Using 3D Retinal Tissue

1) The successfully induced 3D retina tissue was infected using AAV8. 3 days after infection, the GFP expression was observed under the fluorescent microscope. The GFP+3D retinal tissue was collected, and the 3D tissue was digested with papain system to prepare a single-cell suspension. The GFP+ positive retinal cells were screened by flow cytometry to extract the gDNA in accordance with the same protocols as above.

2) PCR reaction

The primers used were shown in Table 26 below in accordance with the same protocols as above.

TABLE 26
Primer sequences

| | | SEQ | PCR |
|---|---|---|---|
| | | ID | product |
| Primer | Primer sequence | NO | length |
| RHO17-forward | CTAGTGTCACCTTGGCCCCTC | 51 | 980 bp |
| primer | | | |
| RHO17-reverse | TGCTGCAAACATGGCCCGAGA | 52 | |
| primer | | | |
| RHO135-forward | TGGCTCCTAGGAGAGGCCCC | 53 | 850 bp |
| primer | | | |
| RHO135-reverse | CTGTTTCTTCTTCTGCCCTA | 54 | |
| primer | | | |

3) T7E1 digestion experiment

The protocols were the same as above.

The results were shown in FIG. 15. FIG. 15A showed the gene editing effects of RHO-SgRNA2 in the 3D retina tissues of patient A and normal person C. A-GFP indicated that the 3D retina of patient A was only infected with pX601-GFP blank control virus; A-17sgRNA2 indicated that the 3D retina of patient A was infected with pX601-R17-GFP-sg2 virus; C-17sgRNA2 indicated that the 3D retina of normal person was transfected with pX601-R17-GFP-sg2 virus. The T7E1 results showed that RHO-17SgRNA2 only had the editing effect on the RHO gene mutation site in the 3D retina of patient A, but had no editing effect at the corresponding gene site in the normal person. FIG. 15B showed the gene editing effects of RHO135-SgRNA1 in the 3D retinal tissues of patient B and normal person C. B-GFP indicated that the 3D retina of patient B was only infected with pX601-GFP blank control plasmid; B-135sgRNA1 indicated that the iPSCs of patient B were infected with pX601-R135-GFP-sg1 virus; C-135sgRNA1 indicated that the 3D retina of normal person was infected with pX601-R135-GFP-sg1 virus. The T7E1 results showed that RHO135-SgRNA1 only had the editing effect on the RHO gene mutation site in the 3D retina of patient B, but had no editing effect at the corresponding gene site in the normal person. The results were consistent with those of the in vitro experiments as above.

### Example 8. Validation of Gene Editing Efficiency in a Humanized Mouse Model

(1) Construction of Humanized Mice

The construction of humanized mice was completed by Beijing Biocytogen Co., Ltd.

1) Preparation of humanized mice

For the human mutation site, the Beijing Biocytogen Gene Biotechnology Co., Ltd. was commissioned to construct the humanized mouse models for RHO. There were two kinds of humanized mouse models, one was a humanized mouse model carrying the RHO gene mutation site (p.Arg135Trp or p.Thr17Met), and the other was a humanized mouse model with the knock-in humanized fragment but without the mutation site.

2) The protocols were as follows:

a) Development of humanized RHO gene point mutant mice carrying mutations (p.Thr17Met or p.Arg135Trp):

\textcircled{1} Design and construction of the gRNA that recognizes the target sequence in accordance with Scheme 1 codetermined by both sides, namely, Targeting strategy-1-EGE-System;

\textcircled{2} Construction of the CRISPR/Cas9 vector for the cleavage of target gene;

\textcircled{3} Activity detection of sgRNA/Cas9;

\textcircled{4} Design and construction of the targeting vector for gene knock-in in accordance with the protocols of Scheme 1 codetermined by both sides, to make a coding region replacement at the genomic level (4.7 kb replaced with 4.9 kb) and simultaneously introduce the mutation site, that is, c.50C>T, p.Thr17Met, or c.403C>T, p.Arg135Trp;

\textcircled{5} In vitro transcription of sgRNA/Cas9 mRNA;

\textcircled{6} Injection of sgRNA/Cas9 mRNA and targeting vector into mouse fertilized eggs;

\textcircled{7} Detection and propagation of F0 generation mice with RHO gene knock-in;

\textcircled{8} Acquisition and genotype identification of F1 generation heterozygous mice with RHO gene knock-in.

b) Development of humanized RHO gene knock-in mice:

\textcircled{1} Design and construction of the sgRNA that recognizes the target sequence in accordance with Scheme 1 codetermined by both sides, namely, Targeting strategy-1-EGE-System in the experimental scheme;

\textcircled{2} Construction of the CRISPR/Cas9 vector for the cleavage of target gene;

\textcircled{3} Activity detection of sgRNA/Cas9;

\textcircled{4} Design and construction of the targeting vector for gene knock-in in accordance with the protocols of Scheme 1, to make a coding region replacement at the genomic level (4.7 kb replaced with 4.9 kb);

\textcircled{5} In vitro transcription of sgRNA/Cas9 mRNA;

\textcircled{6} Injection of sgRNA/Cas9 mRNA and targeting vector into mouse fertilized eggs;

\textcircled{7} Detection and propagation of F0 generation mice with RHO gene knock-in;

\textcircled{8} Acquisition and genotype identification of F1 generation heterozygous mice with RHO gene knock-in.

(2) Feeding and Breeding

1) After obtaining two kinds of humanized mice, the F1 generation heterozygous mice were inbred to obtain a sufficient number of F2 or F3 generation humanized homozygous mice as soon as possible for AAV virus injection, in order to assess the editing efficiency of AAV8-pX601-RHO-SgRNA obtained above.

(3) Genotype Identification of Humanized Mice

The primer pair WT-F/WT-R was designed for the wild gene sequence. When this pair of primers was used for PCR, only the product of the wild type allele could be amplified, while the product of mutant allele could not be amplified. The primer/Mut-R was designed for the humanized RHO gene sequence in mice. When the WT-F/Mut-R primer pair was used for PCR, only the product of mutant allele could be amplified, while the product of the wild type allele could not be amplified.

The primer sequences were shown in Table 27 below:

TABLE 27
Primer sequences

| Primer | Sequence (5′-3′) | Product |
|---|---|---|
| WT-F | GGCAGCAGTGGGATTAGCGTTAGTA | WT: 494 |
| | (SEQ ID NO: 55) | |
| WT-R | TGTGTAGAGGGTGGTGGTGAATCCT | |
| | (SEQ ID NO: 56) | |
| WT-F | GGCAGCAGTGGGATTAGCGTTAGTA | Mut: 349 |
| | (SEQ ID NO: 55) | |
| Mut-R | ACGATCAGCAGAAACATGTAGGCGG | |
| | (SEQ ID NO: 57) | |

The WT-F/WT-R primer pair was mainly used to identify the presence of the wild-type allele, and to determine the specific genotype of the animal in combination with the PCR results of the WT-F/Mut-R primer pair: homozygous/heterozygous/wild-type.

The criteria for genotype determination were shown in Table 28:

TABLE 28
Genotype determination criteria

| | |
|---|---|
| WT-F/Mut-R | WT-F/WT-R |

| Mutant | Wild-type | Mutant | Genotype |
|---|---|---|---|
| Y | N | N | H/H |
| Y | Y | N | H/+ |
| N | Y | N | +/+ |

Notes:
Y: PCR product with the desired length as detected by gel electrophoresis; N: PCR product without the desired length as detected by gel electrophoresis; H/H: Homozygous genotype; H/+: Heterozygous genotype; +/+: Wild-type.

The PCR reaction system and program were shown in “Example 3.” The PCR product was sent for sequencing to detect whether the RHO gene of humanized mice contained the desired knock-in mutation site.

(4) Subretinal Injection of AAV8 Virus in Mice:

The AAV virus injected into the mice was the virus used to infect the 3D retinal tissue.

1) The pupils were dilated with 1% atropine at 30 min before injection, and dilated again before anesthetization.

2) The mice were anesthetized by intraperitoneal injection of 80 mg/kg ketamine+8 mg/kg xylazine. Then the mice were placed in front of the animal experiment platform of the ophthalmic surgery microscope, and a drop of 0.5% proparacaine was dropped on the eyes of mice for local anesthesia. The fluorescein sodium stock solution was added to the AAV virus at a concentration of 100:1, and mixed by low-speed centrifugation.

3) A minipore was pricked by insulin needle in advance in the ciliary pars plana of the mouse eyeball, through which a microsyringe needle passed to enter the vitreous chamber of the mouse eyeball. At this time, an appropriate amount of 2% hydroxymethyl cellulose was dropped on the mouse eyeball such that the mouse fundus could be seen under the microscope. Then the needle was inserted into the contralateral periphery subretinal space while keeping off the lens. The AAV virus with fluorescein sodium was slowly pushed-in, with an injection volume of 1 μl in each eye. The fluorescein sodium served as the indicator for judging whether it was injected into the subretinal space.

4) Whether the mouse was normal or not was observed after injection, and the neomycin eye ointment was applied to prevent infection.

(5) Editing Efficiency Assessment of AAV8-pX601-RHO-SgRNA in Humanized Mice

1) 3 months after injection, the GFP expression in the mouse eyeball was observed using an in vivo imaging system for mouse. The GFP+ mouse eyeball was collected, and the 3D tissue was digested with papain system to prepare a single-cell suspension. The GFP+ positive retinal cells were screened by flow cytometry to extract the gDNA in accordance with the same protocols as above.

2) PCR reaction

The primers used were shown in Table 29 below in accordance with the same protocols as above.

TABLE 29
Primer sequences

| | | SEQ | PCR |
|---|---|---|---|
| | | ID | product |
| Primer | Primer sequence | NO | length |
| RHO17-forward | GAGTGTGGGGACTGGATGAC | 58 | 950 bp |
| primer | | | |
| RHO17-reverse | GGGACTCTCCCAGACCCCTC | 59 | |
| primer | | | |
| RHO135-forward | TGTCCGGGTTATTTCATTTC | 60 | 860 bp |
| primer | CC | | |
| RHO135-reverse | GAGATGGGACCAGCCCTTGT | 61 | |
| primer | | | |

3) T7E1 digestion experiment

The protocols were the same as above.

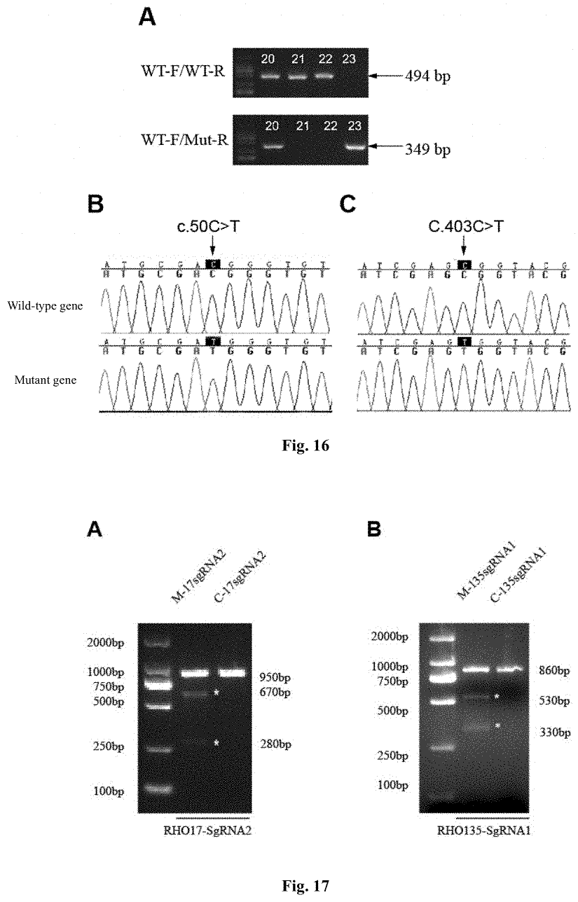

FIG. 16A showed the results of gel electrophoresis for RHO humanized mouse genotype identification. The mouse No. 20 was a humanized mouse carrying the heterozygous mutation, and the mice Nos. 21 and 22 were humanized mice carrying WT. FIG. 16B showed a humanized mouse carrying the homozygous mutation of RHO c.50C>T, and FIG. 16C showed a humanized mouse carrying the homozygous mutation of RHO c.403C>T.

FIG. 17A showed the gene editing efficiency of RHO17-SgRNA2 in humanized mice. M-17sgRNA2 indicated that the humanized mouse carrying the homozygous mutation of c.50C>T was injected with AAV-R17-sg2-SaCas9 virus, while C-17sgRNA2 indicated that the humanized mouse carrying WT was injected with AAV-R17-sg2-SaCas9 virus. The T7E1 results showed that RHO-17SgRNA2 only had the editing effect on the humanized mouse carrying the mutation, but had no editing effect at the corresponding site in the humanized mouse carrying WT. FIG. 17B showed the gene editing efficiency of RHO135-SgRNA1 in humanized mice. M-135sgRNA1 indicated that the humanized mouse carrying the homozygous mutation of c.403C>T was injected with AAV-R135-sg1-SaCas9 virus, while C-135sgRNA1 indicated that the humanized mouse carrying WT was injected with AAV-R135-sg1-SaCas9 virus. The T7E1 results showed that RHO-135SgRNA1 only had the editing effect on the humanized mouse carrying the mutation, but had no editing effect at the corresponding site in the humanized mouse carrying WT. The results of the in vivo experiments were consistent with those of the in vitro experiments as above.

1. A method for treating retinitis pigmentosa, comprising the step of: enabling a subject in need thereof to have a functional RHO gene, wherein the functional RHO gene does not comprise a mutation site selected from the group consisting of c.C50T and c.C403T.

2. The method according to claim 1, comprising the step of: removing the mutation site of the RHO gene in the subject in need thereof, wherein the removing comprises knocking out the mutation site and/or reducing the expression level of the RHO gene comprising the mutation site.

3. (canceled)

4. The method according to claim 2, wherein the removing comprises not affecting the expression level and/or function of the wild-type RHO gene in the subject, wherein the removing comprises realizing a double-strand break in the RHO allele comprising the mutation, and/or the removing comprises administrating to the subject in need thereof at least one vector capable of removing the mutation site.

5-6. (canceled)

7. The method according to claim 4, wherein the vector comprises a sequence encoding a gRNA that specifically binds to the mutation site, wherein the gRNA specifically binds to at least a portion of nucleic acid in the RHO allele comprising the mutation site.

8. (canceled)

9. The method according to claim 7, wherein the gRNA is specifically complementary to at least a portion of nucleic acid sequence in exon 1 of the RHO allele comprising c.C50T mutation, or the gRNA is specifically complementary to at least a portion of nucleic acid sequence in exon 2 of the RHO allele comprising c.C403T mutation.

10. The method according to claim 7, wherein the gRNA comprises a nucleotide sequence set forth in any of SEQ ID NOs. 44-45 and 47.

11. The method according to claim 7, wherein the sequence encoding the gRNA comprises a nucleotide sequence set forth in any of SEQ ID NOs. 1-2 and 4.

12-14. (canceled)

15. The method according to claim 4, wherein the vector comprises a nucleic acid encoding Cas protein.

16. The method according to claim 15, wherein the Cas protein includes Cas9 protein.

17. The method according to claim 15, wherein the sequence encoding the gRNA and the nucleic acid encoding Cas protein are located in a same vector.

18. The method according to claim 4, wherein the vector includes a viral vector.

19. The method according to claim 4, wherein the vector is an adeno-associated virus vector (AAV).

20. The method according to claim 4, wherein the vector is AAV8.

21. The method according to claim 1, wherein the subject includes East Asians.

22. (canceled)

23. The method according to claim 4, wherein the administrating includes injection.

24. The method according to claim 4, wherein the administrating includes injection in the subretinal space.

25-43. (canceled)

44. A composition for treating retinitis pigmentosa in a subject, comprising an active ingredient that removes a mutation site in an RHO gene, and a pharmaceutically acceptable carrier, wherein the mutation site is selected from the group consisting of c.C50T and c.C403T.

45. The composition according to claim 44, wherein the active ingredient comprises a sequence encoding a gRNA that specifically binds to the mutation site.

46. The composition according to claim 45, wherein the gRNA comprises a nucleotide sequence set forth in any of SEQ ID NOs. 44, 45, and 47.

47. The composition according to claim 44, wherein the sequence encoding the gRNA comprises a nucleotide sequence set forth in any of SEQ ID NOs. 1, 2, and 4.

48. The composition according to claim 46, wherein the active ingredient comprises Cas protein.

49. The composition according to claim 48, wherein the Cas protein includes Cas9 protein.

50. The composition according to claim 48, wherein the sequence encoding the gRNA and the nucleic acid encoding Cas protein are located in a same vector.

51. The composition according to claim 44, wherein the vector includes a viral vector.

52. The composition according to claim 44, wherein the vector is an adeno-associated virus vector (AAV).

53. The composition according to claim 44, wherein the vector is AAV8.
